(19) United States
(12) Patent Application Publication
(10) Pub. No.: US 2025/0268816 A1
(43) Pub. Date: Aug. 28, 2025

(54) EXTENDED-RELEASE FORMULATION CONTAINING CRESOL

(71) Applicant: UNIVERSITY OF MARYLAND, BALTIMORE, Baltimore, MD (US)

(72) Inventors: Tao LOWE, Hummelstown, PA (US); Curt I. CIVIN, Baltimore, MD (US)

(21) Appl. No.: 18/857,495

(22) PCT Filed: Apr. 26, 2023
(86) PCT No.: PCT/US2023/019948

§ 371 Date: Oct. 17, 2024

Related U.S. Application Data
(60) Provisional application No. 63/334,938, filed on Apr. 26, 2022.
(60) Provisional application No. 63/334,896, filed on Apr. 26, 2022.

(51) Int. Cl. A61K 9/00; A61K 31/497; A61K 38/28; A61K 47/10; A61K 47/34; A61P 3/10

(52) U.S. Cl. CPC A61K 9/0024; A61K 31/497; A61K 38/28; A61K 47/10; A61K 47/34; A61P 3/10

(57) ABSTRACT

This invention relates generally to injectable drug delivery systems and methods of preparing same, and in particular to drug delivery systems comprising biodegradable polymeric nanogel formulations that form a depot (gel) in the body once injected, and can release therapeutic agents with enhanced extended-release times in the subject being treated. The polymer matrix drug delivery systems of the subject invention are comprised of a biodegradable polymer, a solvent or a combination of solvents, an alkylphenol, and therapeutic agents such as insulin, tetrandrine, other small molecule drugs (preferably hydrophobic drugs) to treat a variety of diseases including diabetes and hearing loss. A preferred alkylphenol is meta cresol which has demonstrated importance for dissolving and stabilizing the therapeutic agents, and forming gels with better shape and controlled drug release after injection.

### CROSS-REFERENCE TO RELATED APPLICATIONS

This application is a 371 National Stage application of PCT Application PCT/US2023/019948, filed 26 Apr. 2023, which claims the benefit of U.S. provisional application Ser. No. 63/334,938, filed 26 Apr. 2022 and U.S. provisional application Ser. No. 63/334,896, filed 26 Apr. 2022. The entire contents of these applications are hereby incorporated by reference as if fully set forth herein.

### BACKGROUND

### 1. Field of the Invention

This invention relates generally to injectable in situ gelling, biodegradable drug delivery systems, and in particular to drug delivery systems comprising cresol providing enhanced extended-release times of therapeutic agents in the subject being treated.

### 2. Background of the Invention

Therapeutic agents in the form of chemically synthesized drugs and biologics manufactured in living things play important roles for treating all types of diseases and for tissue and organ regeneration. Such treatments and regenerations are used for various conditions including but not limited to neurological, eye, brain, ear, temporomandibular, dental, oral, facial, blood, bone, cartilage, heart, lung, skin, muscle, reproductive, cancer, and diabetes diseases, and also including injuries, fractures and/or other damage to tissues and organs.

Diabetes mellitus, commonly known as diabetes, is an endocrine disorder characterized by persistently high blood glucose levels over a prolonged period that can damage nerves and blood vessels and related organs. It currently has no cure and is one of the major global health problems with a significant economic burden. It affects approximately 537 million adults (20-79 years, 6.7%) worldwide, and its prevalence is expected to increase to 643 million by 2030 and 783 million by 2045. According to the United States Centers for Disease Control (CDC) National Diabetes Statistics Report published in 2020, nearly 37.3 million Americans, i.e., approximately 11.3% of the U.S. population, have diabetes, and among which about 5% of the patients have type 1 diabetes and 95% of the patients are estimated to have type 2 diabetes. The global economic burden of diabetes was estimated to be $1.3 trillion in 2015. In the U.S., the total cost of diagnosed diabetes was estimated to be $327 billion in 2017, including $237 billion for direct medical costs and the rest for reduced productivity of the diabetic patients.

Type 1 diabetes is characterized by pancreatic β-cell destruction leading to absolute insulin deficiency; whereas type 2 diabetes is characterized by insulin resistance with a progressive deficiency in insulin secretion by pancreatic B-cells. Both type 1 and type 2 diabetes manifest persistent elevation of blood glucose level. However, if untreated, both type 1 and type 2 diabetes can cause serious complications including stroke, myocardial infarction, vision loss, amputation, chronic kidney diseases, and mortality. For patients with type 1 diabetes, multiple daily injections of insulin are the only treatment option. For patients with type 2 diabetes, the treatment starts with management of diabetes through change in lifestyle including healthy eating, weight loss and regular exercise followed by pharmacological intervention with oral metformin monotherapy. If normoglycemia is still not achieved, metformin is given in combination with the following classes of small molecule oral medications: sulfonylurea, thiazolidinedione, DPP-4 inhibitor and SGLT2 inhibitor. However, these oral medications often fail to achieve desired glucose lowering effect, and insulin therapy is eventually included in the treatment regimen when the hemoglobin A1c (HbA1c) level of type 2 diabetes patients is consistently more than 6.5%. Given the prevalence of type 2 diabetes, type 2 diabetic patients account for the use of the majority of insulin in the market.

Infectious Diseases (such as HIV/AIDS and COVID19) affect both adults and children with 38.4M cases and 650,000 deaths in the world in 2021 and 1.2M cases and 15,815 deaths in the United States in 2019. The global infectious disease market was valued at $30.89 billion in 2021, and is projected to reach $53.08 billion by 2030 at a compound annual growth rate (CAGR) of 6.2%. As of April 2023, over 762 million people have been diagnosed with COVID-19 cases and over 6.8 million of those cases resulted in mortality. Vaccination has significantly impacted the COVID-19 epidemic in reducing cases and deaths. However, 2.3 billion people still remain unvaccinated and 89% of these people are located in developing countries.

Leukemia is an umbrella term for various devastating blood cancers that affect all ages, with 470,000 and 61,000 newly diagnosed cases and about 300,000 and 23,000 deaths annually in the world and USA, respectively. The total cost for the treatment of leukemias in USA was $8.7B in 2015 and is expected to increase to $13B by 2030. Acute myeloid leukemia (AML) is a type of leukemia that is a cancer of bone marrow that results from impaired differentiation and proliferation of myeloid cells. This disease has a high death rate in the United States, with over 11,000 deaths so far in 2022, and increasing costs for treatment.

Many therapeutic agents have short half-lives from a few minutes to one month necessitating creative methods for achieving their extended release in the subject once administered. In situ controlled release of such therapeutic agents offers many advantages and avoids certain disadvantages associated with traditional drug delivery methods. For example, in situ rate-controlled drug administration avoids the variability in absorption and metabolism associated with oral therapy. It further provides continuity of drug administration, permitting the use of a pharmacologically active agent with a short biological half-life. Moreover, there is less chance of over- or under-dosing with an in situ drug delivery regimen, and patient compliance with a multi-day, -week, or -month in situ drug delivery regimen is superior to frequent oral dosing.

Currently, methods used for the sustained release of therapeutic agents in situ include their deployment in hydrogels, implants, pumps, microparticles and nanoparticles, all of which can suffer from various shortcomings and limitations. Hydrogels and implants, for instance, require surgical implantation and removal (if the biomaterials are not biodegradable), which is costly and invasive. Pumps have the disadvantages of being clunky, of requiring the user to carry extra batteries or a charger, risk of battery failure, and infection risk. Existing microparticles and nanoparticles, including polymeric micro/nanoparticles made of poly(lactic acid-co-glycolic acid) (PLGA)/poly(lactic acid) (PLA), liposomes, dendrimers, polymeric micelles, inorganic and carbon nanotubes, for instance, either cannot achieve more than one month of sustained release of the therapeutic agent or need to use organic solvents for biologic encapsulation which can denature biologics. There is a need in the art for new technologies for administration of therapeutic agents, particularly with longer sustained release times and therapeutic stability.

### SUMMARY OF THE INVENTION

In particular embodiments, the present invention relates to compositions and methods for providing a flexible or flowable biodegradable composition that can form a gel in situ and be used in vivo for the release of therapeutically effective amounts of a variety of therapeutics. There is also a continuing need for these compositions to allow for the sustained release of biologics and chemically synthesized drugs over a significantly longer term, and without degradation, than was heretofore possible. Accordingly, provided herein is a delivery system for therapeutic agents, and methods for using the delivery system, to deliver an active therapeutic agent to a subject in need thereof on an enhanced extended release basis.

In one aspect, provided herein is an injectable polymer matrix drug delivery system comprising: a) a biodegradable polymer or combinations thereof; b) a solvent or a combination of solvents; c) an alkylphenol; and d) a therapeutic agent.

In some embodiments, the therapeutic agent is insulin or an insulin analog. For instance, in certain embodiments, the present invention relates to injectable and non-toxic formulations that can continuously release insulin, an anabolic peptide hormone with a short half-life of about 4-6 minutes, at 12-24 Unit/day to lower blood glucose levels for two weeks after a single Sub-Q injection. As such, these formulations can have significant impact on the treatment of type 1 and type 2 diabetes that can maintain insulin basal level without adverse effects, reduce frequent administration of insulin, improve adherence, and ultimately help management of diabetes in patients around the world.

In some embodiments, the therapeutic agent is tetrandrine. For instance, in certain embodiments, the present invention relates to injectable and non-toxic formulations that can continuously release tetrandrine, a natural bisbenzylisoquinoline alkaloid, for a very broad-spectrum of distinct pharmacological activities, including anticancer, anti-inflammatory, anti-nociceptive, anti-fibrotic, anti-depressant, anti-rheumatoid arthritis, anti-adipogenic, anti-microbial, neuroprotective and memory-improving activities.

In some embodiments, the therapeutic agent is dexamethasone. For instance, in certain embodiments, the present invention relates to injectable and non-toxic formulations that can continuously release dexamethasone, a widely used corticoid that works on the immune system to help relieve swelling, redness, itching, and allergic reactions. The dexamethasone-containing formulations can be used to treat conditions such as arthritis, asthma, skin diseases, eye problems, breathing problems, bone marrow problems, kidney problems, cancers, hearing loss, immune system disorders, blood/hormone disorders, bowel disorders, adrenal gland disorders, Cushing's syndrome, flare-ups of multiple sclerosis, and inflammation response and mortality associated with COVID-19 cytokine storm.

In some embodiments, the therapeutic agent is Remdesivir. For instance, in certain embodiments, the present invention relates to injectable and non-toxic formulations that can continuously release Remdesivir, a nucleoside antiviral agent that has shown clinical effect in reducing ventilation time and hospitalization, is the only anti-viral agent fully FDA approved for treating COVID-19 patients, is originally developed to treat hepatitis C, and is currently under clinical trials for Ebola/Marburg/HIV/AIDS and other infectious diseases. Its half-life is 0.89 hour and it is used as tablets and solutions dosage forms via oral and intravenous daily injection. Injectable long-acting remdesivir is needed for improved efficacy, patient compliance and low cost.

In some embodiments, the therapeutic agent is GS-441524. For instance, in certain embodiments, the present invention relates to injectable and non-toxic formulations that can continuously release GS-441524, a metabolite of remdesivir and a nucleoside analog antiviral drug, has a similar antiviral effect to remdesivir, but with much lower manufacturing cost. GS-441524 is the main plasma metabolite of remdesivir, and has a half-life of around 24 hours in human patients. Remdesivir and GS-441524 were both tested against feline infectious peritonitis (FIP) in cell culture and found to be equivalent. However, both drugs require daily intravenous administration for therapeutic effect, which can be difficult for patients in developing countries to receive. It is still highly desirable to develop a drug delivery system to sustain release GS-441524 to treat viral diseases.

In some embodiments, the therapeutic agent is artemisinin or derivatives. For instance, in certain embodiments, the present invention relates to injectable and non-toxic formulations that can continuously release artemisinin, or its derivative ART838. Artemisinin (ART), discovered in 1971 by Dr. Youyou Tu, is an extract from the sweet wormwood plant that has antileukemic effects. ART and its derivatives have been known as a useful drug for malaria treatment without toxicity. ART and many ART derivatives are hydrophobic and also are known to have efficacy against cancer cells, including leukemia. In particular, several 2-carbon linked dimeric artemisinin analogs (2C-ARTs (see U.S. Pat. No. 9,487,538) were found to effectively kill 9 human leukemia cell lines at half maximal inhibitory concentrations (IC₅₀)<50 nM. However, there are major challenges to several of the currently available 2C-ARTs; their limited water solubility and low bioavailability. Additionally, 2C-ARTs characteristically have short in vivo half-lives, which require frequent dosing to maintain pharmacologically active plasma concentrations. Thus, to improve half-life, novel dimeric ART analogs, such as ART838, have been developed. ART838 has high antileukemic potency and efficacy in vitro and in vivo, but still a half-life of only 3.19 hours in mice. In addition, many ART derivatives have low water solubility and consequently low bioavailability, which are also disadvantages of many other hydrophobic drugs.

In some embodiments, the therapeutic agent is a protein, peptide, antibody or biologic.

In some embodiments, the therapeutic agent is a small molecule drug.

In some embodiments, the therapeutic agent is hydrophobic.

In some embodiments, the alkylphenol is m-cresol.

In another aspect, the subject invention is a method of treating diabetes mellitus, the method comprising administering to a subject in need thereof the injectable polymer matrix drug delivery system described herein.

In still another embodiment, provided herein is a method of reducing blood glucose levels, the method comprising administering to a subject in need thereof the injectable polymer matrix drug delivery system described herein.

In still another aspect, the subject invention is a method of forming a polymer matrix drug delivery system described herein, the method comprising: a) adding a therapeutic agent to an alkylphenol and a solvent or a combination of solvents in a first syringe; b) adding a biodegradable polymer selected from the group consisting of poly(lactic-co-glycolic acid), poly(lactic acid), poly(ε-caprolactone), poly(ethylene glycol-block-lactic acid), poly(alkylcyanoacrylate), polyanhydride, poly(bis(p-carboxyphenoxy) propane-sebacic acid), polyorthoester, polyphosphoester, polyphosphazene, polyurethane, and poly(amino acid), or combinations thereof to a second syringe; c) sterilizing the two syringes by gamma irradiation; and d) mixing the components of the two syringes at the time of injection into the subject requiring treatment.

In some embodiments, the therapeutic agent in the foregoing method is insulin or an insulin analog. In other embodiments, the therapeutic agent is anti-inflammation, anti-infection, anti-fungal, anti-cancer, anti-nociceptive, anti-fibrotic, anti-depressant, anti-rheumatoid arthritis, anti-adipogenic, anti-microbial, anti-viral, anti-HIV, anti-malarial, anti-apoptotic and/or neuroprotective including but not limited to tetrandrine, dexamethasone, remdesivir, GS-441524, artemisinin, ART838, an artemisinin derivative, gilteritinib, baricitinib, venetoclax, sorafenib, islatravir, emtricitabine, tenofovir, tenofovir disoproxil fumarate, tenofovir alafenamide, abacavir, didanosine, lamivudine, stavudine, zidovudine, bictegravir, dolutegravir, elvitegravir, raltegravir, ulonivirine, doravirine, lenacapavir, zanamivir, valaciclovir hydrochloride, acyclovir, lamivudine, indinavir sulfate, nelfinavir, nevirapine, efavirenz, rilpivirine, delavirdine, atazanavir, darunavir, lopinavir and cabotegravir, or a combination thereof.

In particular, the present invention relates to an injectable polymer matrix drug delivery system comprising: a) a biodegradable polymer selected from the group consisting of polyester, poly(lactic-co-glycolic acid), poly(lactic acid), poly(ε-caprolactone), poly(ethylene glycol-block-lactic acid), poly(alkylcyanoacrylate), polyanhydride, poly(bis(pcarboxyphenoxy) propane-sebacic acid), polyorthoester, polyphosphoester, polyphosphazene, polyurethane, and poly(amino acid), or combinations thereof; b) a solvent or a combination of solvents; c) an alkylphenol; and d) a drug.

In preferred embodiments, the biodegradable polymer is selected from poly(lactic-co-glycolic acid), poly(lactic acid), and poly(ε-caprolactone), or combinations thereof.

In preferred embodiments, the solvent is selected from N-methyl-2-pyrrolidone (NMP), benzyl benzoate (BB), benzyl alcohol (BA), triethyl citrate (TEC), acetyl triethyl citrate (ATEC), ethyl acetate (EA), acetyl tributyl citrate (ATBC), dimethyl sulfoxide (DMSO), and any combination thereof.

In preferred embodiments, the biodegradable polymer is selected from poly(L-lactic acid) and poly(D,L-lactic acid), or combinations thereof.

In preferred embodiments, the alkylphenol is a cresol, and most preferably is m-cresol.

In preferred embodiments, the drug is selected from the group consisting of insulin, an insulin analog, tetrandrine, dexamethasone, remdesivir, GS-441524, artemisinin, ART838, an artemisinin derivative, gilteritinib, and any combination thereof. Most preferably, the drug is insulin, an insulin analog, or a combination of insulin and gilteritinib.

In preferred embodiments, the system further comprises zinc ions.

In preferred embodiments, the solvent is selected from the group consisting of N-methyl-2-pyrrolidone (NMP), benzyl benzoate (BB), benzyl alcohol (BA), triethyl citrate (TEC), acetyl triethyl citrate (ATEC), ethyl acetate (EA) dimethyl sulfoxide (DMSO), and a combination thereof; the biodegradable polymer is selected from the group consisting of poly(L-lactic acid), poly(D,Llactic acid), and a combination thereof, and the active pharmaceutical ingredient is selected from the group consisting of insulin, an insulin analog, tetrandrine, dexamethasone, remdesivir, GS-441524, artemisinin, ART838, an artemisinin derivative, gilteritinib, and any combination thereof.

In additional embodiments, solvent is a combination of NMP and TEC; NMP and ATEC; NMP and ATBC; NMP and BB; NMP and BA; NMP and EA; TEC and BB; ATEC and BB; ATBC and BB; TEC and BA; ATEC and BA; ATBC and BA; TEC and EA; ATEC and EA; ATBC and EA; NMP, TEC and BB; NMP, ATEC and BB; NMP, ATBC and BB; NMP, TEC and BA; NMP, ATEC and BA; NMP, ATBC and BA; NMP, TEC and EA; NMP, ATEC and EA; NMP, ATBC and EA; TEC, BB and EA; ATEC, BB and EA; ATBC, BB and EA; TEC, BA and EA; ATEC, BA and EA; ATBC, BA and EA; NMP and DMSO; BB and DMSO; BA and DMSO; TEC and DMSO; ATEC and DMSO; ATBC and DMSO; EA and DMSO; NMP, TEC and DMSO; NMP, ATEC and DMSO; NMP, ATBC and DMSO; NMP, BB and DMSO; NMP, BA and DMSO; NMP, EA and DMSO; TEC, ATEC and DMSO; TEC, BB and DMSO; TEC, BA and DMSO; TEC, EA and DMSO; ATEC, BB and DMSO; ATEC, BA and DMSO; ATEC, EA and DMSO; BB, BA and DMSO; BB, EA and DMSO; or BA, EA and DMSO. Preferably, the solvent combination is NMP and TEC, NMP and ATEC, NMP and BB, NMP and BA, or DMSO and TEC.

In preferred embodiments, the drug delivery system comprises the polymer in about 0-50% by weight, the solvent in about 50-95% by weight, the alkylphenol in about 0.1-50% by weight, and insulin or an insulin analog in about 0.130% by weight.

In preferred embodiments, the drug delivery system is packaged in two syringes comprising: a) one syringe containing the polymer solution, b) one syringe containing a solvent or a combination of solvents, the alkylphenol, and the drug. Preferably, the drug delivery system is formulated for subcutaneous injection or intramuscular injection.

In additional embodiments, the invention relates to a method of treating diabetes mellitus in a subject in need thereof, the method comprising administering to a subject in need thereof the injectable polymer matrix drug delivery system of any of the embodiments described herein. Preferably, the diabetes mellitus is type 1 diabetes or type 2 diabetes.

In additional embodiments, the invention relates to a method of reducing blood glucose levels in a subject in need thereof, the method comprising administering to a subject in need thereof the injectable polymer matrix drug delivery system of the embodiments described herein.

In additional embodiments, the invention relates to a method of treating a condition selected from the group consisting of inflammation, infection, cancer, hearing loss, COVID19, and HIV in a subject in need thereof, the method comprising administering to a subject in need thereof the injectable polymer matrix drug delivery system of the embodiments described herein.

In preferred embodiments, upon administration to the subject in need thereof, the active pharmaceutical ingredient is released for at least 140 days or at least 5 days.

In preferred embodiments, the drug delivery system forms a semisolid or solid depot at the injection site.

In additional embodiments, the invention relates to a method of administering to a subject the polymer matrix drug delivery system of any one of claims 1-17 comprising: a) adding insulin or an insulin analog to an alkylphenol and a solvent or a combination of solvents in a first syringe; b) adding a biodegradable polymer selected from the group consisting of poly(lactic-co-glycolic acid), poly(lactic acid), poly(ε-caprolactone), poly(ethylene glycol-block-lactic acid), poly(alkylcyanoacrylate), polyanhydride, poly(bis(p-carboxyphenoxy) propane-sebacic acid), polyorthoester, polyphosphoester, polyphosphazene, polyurethane, and poly(amino acid), or combinations thereof to a second syringe; c) sterilizing the two syringes by gamma irradiation; d) mixing the components of the two syringes at the time of injection; and e) injecting the mixed components into the subject.

In additional embodiments, the invention relates to a method of preparing the polymer matrix drug delivery system as described herein comprising: a) adding insulin or an insulin analog to an alkylphenol and a solvent or a combination of solvents in a composition; and b) adding a biodegradable polymer selected from the group consisting of poly(lactic-co-glycolic acid), poly(lactic acid), poly(ε-caprolactone), poly(ethylene glycol-block-lactic acid), poly(alkylcyanoacrylate), polyanhydride, poly(bis(pcarboxyphenoxy) propane-sebacic acid), polyorthoester, polyphosphoester, polyphosphazene, polyurethane, and poly(amino acid), or combinations thereof to the composition.

### BRIEF SUMMARY OF THE DRAWINGS

Certain embodiments are illustrated by way of example, and not by way of limitation, in the figures of the accompanying drawings. However, the invention is not limited to the precise arrangements and instrumentalities of the embodiments depicted in the drawings.

FIG. 1 is a graph showing sustained release of insulin over sixteen days upon injection of the drug delivery systems described herein.

FIG. 2 is a graph showing insulin release profiles for formulations with and without m-cresol.

FIG. 3 is a set of photographs showing the morphology and stability of insulin-loaded depot in buffer solutions over twenty hours for formulations with and without m-cresol.

FIG. 4 is a set of photographs showing the morphology and stability of insulin-loaded depot in buffer solutions over forty hours for formulations with and without m-cresol.

FIG. 5A and FIG. 5B are graphs showing the effect of insulin loading on the cumulative release of insulin from ISFGSs.

FIG. 6A and FIG. 6B are graphs showing the effect of insulin loading and polymer intrinsic viscosity on the cumulative release of insulin from ISFGSs.

FIG. 7A and FIG. 7B are graphs showing the effect of polymer intrinsic viscosity on the cumulative release of insulin from ISFGSs.

FIG. 8A and FIG. 8B are graphs showing the effect of polymer intrinsic viscosity on the cumulative release of insulin from ISFGSs.

FIG. 9A and FIG. 9B are graphs showing the effect of zinc amount on the cumulative release of insulin from ISFGSs.

FIG. 10A and FIG. 10B are graphs showing the effect of zinc amount on the cumulative release of insulin from ISFGSs.

FIG. 11A and FIG. 11B are graphs showing the effect of formulation volume on the cumulative release of insulin from the ISFGSs.

FIG. 12A and FIG. 12B are graphs showing the effect of insulin type on the cumulative release of insulin from ISFGSs containing insulin and zinc at insulin:zinc at 1:28 w:w. Insulin was released continuously for 7 days and slower from the formulation containing Degludec than regular insulin.

FIG. 13A and FIG. 13B are graphs showing that subcutaneously injected insulin decreased the blood glucose level in diabetic rats. Higher insulin dose at 5 IU decreased the blood glucose level to about 100 mg·dL⁻¹ within 4 hours after the injection.

FIG. 14A, FIG. 14B, and FIG. 14C are graphs showing that subcutaneously injected ISFGSs containing 42 IU insulin decreased the blood glucose level in diabetic rats for 5 days with fluctuation.

FIG. 15A, FIG. 15B, and FIG. 15C are graphs showing that subcutaneously injected ISFGSs containing 42 IU insulin decreased the blood glucose level in diabetic rats for 78 hours with fluctuation.

FIG. 16A, FIG. 16B, and FIG. 16C are graphs showing that subcutaneously injected ISFGSs containing 42 IU insulin decreased the blood glucose level in diabetic rats for 30 hours.

FIG. 17A, FIG. 17B, and FIG. 17C are graphs showing that subcutaneously injected ISFGSs containing 42 IU insulin decreased the blood glucose level in diabetic rats for 7.25 days.

FIG. 18A, FIG. 18B, and FIG. 18C are graphs showing that subcutaneously injected ISFGSs containing 42 IU insulin decreased the blood glucose level in diabetic rats for 9 days with fluctuation.

FIG. 19A, FIG. 19B, and FIG. 19C are graphs showing is a graph showing that subcutaneously injected ISFGSs containing 42 IU insulin decreased the blood glucose level in diabetic rats for 9 days with fluctuation.

FIG. 20A, FIG. 20B, and FIG. 20C are graphs showing that subcutaneously injected ISFGSs containing 42 IU insulin decreased the blood glucose level in diabetic rats for 6.25 days with fluctuation.

FIG. 21A, FIG. 21B, and FIG. 21C are graphs showing that subcutaneously injected ISFGSs containing 42 IU insulin decreased the blood glucose level in diabetic rats for 8.25 days with fluctuation.

FIG. 22A, FIG. 22B, FIG. 22C, and FIG. 22D are graphs showing the effect of injection amount and polymer content on the blood glucose level lowering after subcutaneously injection of ISFGSs.

FIG. 23A, FIG. 23B, FIG. 23C, and FIG. 23D are graphs showing the effect of PLGA intrinsic viscosity and addition of chitosan or zinc on the blood glucose level lowering after subcutaneously injection of ISFGSs.

FIG. 24A and FIG. 24B are graphs showing the effect of polymer content on the blood glucose level lowering after subcutaneously injection of ISFGSs.

FIG. 25A and FIG. 25B are graphs showing the effect of PLGA on the blood glucose level lowering after subcutaneously injection of ISFGSs.

FIG. 26A and FIG. 26B are graphs showing the effect of PLGA and PLA on the blood glucose level lowering after subcutaneously injection of ISFGSs.

FIG. 27 is a graph showing the results of a tetrandrine release study.

FIGS. 28A and 28B are photographs showing the morphology and stability of tetrandrine depots in buffer solutions over 1 day for formulations with and without m-cresol, respectively.

FIG. 29 is a graph showing the results of a dexamethasone release study using 6 wt % Dex and 24 wt % Polymer 80 μL.

FIGS. 30A and 30B are graphs showing the effect of polymer composition on the cumulative release of remdesivir from ISFGSs; 3 wt % 80 and 160 uL-24 wt %.

FIGS. 31A and 31B are graphs showing the effect of polymer composition on the cumulative release of GS-441524 from 80 μL of ISFGSs.

FIGS. 32A and 32B are graphs showing the effect of polymer composition on the cumulative release of GS-441524 from 160 μL of ISFGSs.

FIG. 33A and FIG. 33B are a set of graphs showing the effect of polymer composition and solution volume on the cumulative release of artemisinin from ISFGSs.

FIG. 34 shows depot appearance results.

FIG. 35 shows ART838 release results.

FIG. 36A through FIG. 36D are micrographs showing depot morphology. FIG. 36A: 3 days, FIG. 36B: 10 days; FIG. 36C: 20 days, FIG. 36D: 28 days.

FIGS. 37A and 37B are graphs showing the cumulative release of gilteritinib from ISFGSs: Gilteritinib 4 wt % Release 80 μL PLGA 50/50 depots.

### DETAILED DESCRIPTION OF EMBODIMENTS OF THE INVENTION

### 1. Overview

Provided herein is an injectable drug delivery system, comprising a biodegradable polymer, a solvent, or combination of solvents, and an active therapeutic agent. Also provided herein are methods related to administration of the injectable drug delivery system described herein.

The biodegradable polyesters in the formulations used in the drug delivery system will gradually completely degrade on site after injection. Owing to the complete degradation of the delivery system, no polymers will accumulate in the body and no surgical removal will be required. The drug delivery system can be used by lower-level health care providers and may even be self-administered by subjects, in need thereof, by simple subcutaneous or intramuscular injection.

### 2. Definitions

Unless defined otherwise, all technical and scientific terms used herein have the same meaning as commonly understood by one of ordinary skill in the art. Generally, the nomenclature used herein and the laboratory procedures in cell culture, molecular genetics, organic chemistry, and peptide chemistry are those well-known and commonly employed in the art. Although various methods and materials similar or equivalent to those described herein can be used in the practice or testing of the present invention, suitable methods and materials are described below. However, the skilled artisan understands that the methods and materials used and described are examples and may not be the only ones suitable for use in the invention. Moreover, as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of plus or minus 20 percent of the recited value, so that, for example, “about 0.125” means 0.125±0.025, and “about 1.0” means 1.0±0.2. Notwithstanding that the numerical ranges and parameters setting forth the broad scope of the invention are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “alkyl,” by itself or as part of another substituent means, unless otherwise stated, a straight or branched chain hydrocarbon having the number of carbon atoms designated (i.e., C1-C6-alkyl means an alkyl having one to six carbon atoms) and includes straight and branched chains. In an embodiment, C1-C6 alkyl groups are provided herein. Examples include methyl, ethyl, propyl, isopropyl, butyl, isobutyl, tert butyl, pentyl, neopentyl, and hexyl.

As used herein, the term “alkylphenol” refers to an organic reagent having a benzene core structure functionalized with 1-5 alcohol (—OH) substituents and 1-5 C1-C6 alkyl substituents and includes straight and branched chains. In an embodiment, alkylphenols are provided herein. Examples include cresols: m-cresol, o-cresol, and p-cresol.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

The term “polymer”, as used herein, refers to a polymeric compound prepared by polymerizing monomers, whether of the same or a different type. The term “polymer” thus comprises, homopolymers, copolymers, block copolymers. The term “homopolymer” refers to polymers prepared from only one type of monomer. The term “copolymer”, as used herein, refers to polymers prepared by the polymerization of at least two different types of monomers. Preferably the polymer is a biocompatible and/or biodegradable polymer. The term “biocompatible” is used herein to refer to polymers and any metabolites or degradation products thereof that are generally non-toxic to the recipient and do not cause any significant adverse effects to the subject. The term “biodegradable” is used herein to mean capable of being broken down into innocuous metabolites or degradation products in the normal functioning of the body and which are capable of being metabolized, eliminated, or excreted by the subject. The degradation time is a function of the polymer composition and morphology. Suitable degradation times are from days to weeks. For example, the polymer may degrade over a time period from 1 day to 3 years, preferably 1 day to 2 years, more preferably from 1 day to 1 year, most preferably from 1 day to 36 weeks.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “solvent” refers to an organic compound or a non-organic compound capable of dissolving a solute. Solvents described herein may be non-polar, semi-non-polar, semi-polar, or polar. In preferred embodiments described herein, the solvents are semi-non-polar, semi-polar, or polar. In other preferred embodiments described herein, the solvents are semi-polar or polar. One example of a non-polar solvent is pentane, and one example of a polar solvent is water.

As used herein, the term “subject,” “individual,” or “patient” refers to a human or a non-human mammal. Non-human mammalian subjects include, for example, a mouse, a rabbit, a rat, a transgenic non-human animal, a domestic animal such as a dog or a cat, or farmed animals such as a cow, a horse, a pig, a sheep or a goat, and marine mammals such as a dolphin. Preferably, the patient, subject, or individual is human.

As used herein, the term “substituted” means that an atom or group of atoms has replaced hydrogen as the substituent attached to another group.

As used herein, the phrase “therapeutic agent” (also referred to herein as “active pharmaceutical ingredient”) refers to a substance used in the diagnosis, cure, mitigation, treatment or prevention of disease, or to have direct effect in restoring, correcting or modifying physiological functions in a subject. Therapeutic agents include, generally, both drugs manufactured through chemical synthesis, and biologics manufactured in a living system or artificially. More specifically, therapeutic agents include, pharmaceutically active compounds, hormones, growth factors, enzymes, DNA, plasmid DNA, RNA, siRNA, viruses, proteins, peptides, lipids, pro-inflammatory molecules, antibodies, antibiotics, anti-inflammatory agents, anti-sense nucleotides and transforming nucleic acids or combinations thereof. Any of the therapeutic agents may be combined to the extent such combination is biologically compatible. By varying the drug's administered dosage, the effect in a subject may vary.

In certain embodiments, the therapeutic agent is insulin. As used herein, the term “insulin” refers to a natural peptide hormone made by the pancreas that controls the level of the sugar glucose in the blood. Insulin permits cells to use glucose.

In certain embodiments, the therapeutic agent is an insulin analog. As used herein, the term “insulin analog” refers to human insulin in which one or more amino acid residues have been replaced by another amino acid residue or deleted or in which the A chain and/or the B chain has been extended by addition of one or more amino acid residues at the N-terminal or at the C-terminal and which controls the level of glucose in the blood but with different pharmacokinetics than the naturally occurring insulin. It is also noted that “insulin analog” as used herein, includes pre-insulin, insulin prodrugs, insulin derivatives, recombinant insulin, insulin salts, insulin complexes, or insulin from any origin, or any acceptable form thereof which have activity similar to native insulin. A preferred insulin analog is “insulin lispro,” a rapid acting insulin analog, which is marketed under the trade name HUMALOG®. It is engineered through recombinant DNA technology so that the penultimate lysine and proline residues on the C-terminal end of the B-chain are reversed. This modification does not alter the insulin receptor binding, but blocks the formation of insulin dimers and hexamers. This allows larger amounts of active monomeric insulin to be available for postprandial (after meal) injections.

The term “treat,” “treated,” “treating,” or “treatment” includes the diminishment or alleviation of at least one symptom associated or caused by the state, disorder or disease being treated. In certain embodiments, the treatment comprises administering the drug delivery systems provided herein for conditions related to diabetes mellitus.

### 3. Embodiments of the Invention

### A. Polymer Matrix Drug Delivery System

In one aspect, the subject invention relates to an injectable polymer matrix drug delivery system (also referred to herein more simply as a “drug delivery system”) comprised of four primary components, namely: 1) a biodegradable polymer or a combination thereof; 2) a solvent or a combination of solvents; 3) an alkylphenol; and 4) a therapeutic agent. Each of these components will now be described in detail.

### 1) The Polymer

The first primary component is a biodegradable polymer selected from the group consisting of polyester, poly(lactic-co-glycolic acid) (PLGA), poly(lactic acid), poly(ε-caprolactone), poly(ethylene glycol-block-lactic acid), poly(alkylcyanoacrylate), polyanhydride, poly(bis(p-carboxyphenoxy) propane-sebacic acid), polyorthoester, polyphosphoester, polyphosphazene, polyurethane, and poly(amino acid), or combinations thereof, either among themselves or their copolymers and/or blends with poly(ethylene glycol) (PEG).

The biodegradable polymer may be PLGA in some embodiments. PLGA is a biocompatible and biodegradable co-polymer of lactic acid and glycolic acid, and various forms of PLGA are characterized by the ratio of lactic acid:glycolic acid. Lactic acid can be L-lactic acid, D-lactic acid, or D,L-lactic acid. The degradation rate of PLGA can be adjusted by altering the lactic acid-glycolic acid ratio. In some embodiments, PLGA to be used in accordance with the methods and systems described herein is characterized by a lactic acid:glycolic acid ratio of approximately 90:10, approximately 85:15, approximately 75:25, approximately 60:40, approximately 50:50, approximately 40:60, approximately 25:75, approximately 15:85, or approximately 10:90.

Similarly, the degradation rate of combinations of biodegradable polymers can be adjusted by altering the relative ratios of the polymers. Thus, in some embodiments, combinations to be used in accordance with the methods and systems described herein are characterized by ratios of approximately 90:10, approximately 85:15, approximately 75:25, approximately 60:40, approximately 50:50, approximately 40:60, approximately 25:75, approximately 15:85, or approximately 10:90. For example, provided herein are combinations of poly(lactic acid) and poly(lactic-co-glycolic acid) in ratios of approximately 5:1, 4:1, and 1:1.

In one embodiment, the biodegradable polymer of the drug delivery system described herein is selected from poly(lactic-co-glycolic acid), poly(lactic acid), and poly(ε-caprolactone), or combinations thereof.

In another embodiment, the biodegradable polymer of the drug delivery system described herein is selected from poly(L-lactic acid) and poly(D,L-lactic acid), or combinations thereof.

In another embodiment, the drug delivery system described herein comprises the polymer in about 0-50% by weight, the solvent in about 50-95% by weight, the alkylphenol in about 0.1-50% by weight, and insulin, an insulin analog, tetrandrine, dexamethasone, remdesivir, GS-441524, artemisinin, ART838, an artemisinin derivative, or gilteritinib, or a combination thereof in about 0.1-30% by weight.

In still another embodiment, the drug delivery system described herein comprises poly(lactic-co-glycolic acid) in about 2% by weight, poly(lactic acid) in about 10% by weight, N-methyl-2-pyrrolidone (NMP) and triethyl citrate (TEC) (9/1 w/w) in about 57% by weight, m-cresol in about 30% by weight, and insulin, an insulin analog, or an anti-inflammation, anti-infection, anti-fungal, anti-cancer, anti-nociceptive, anti-fibrotic, anti-depressant, anti-rheumatoid arthritis, anti-adipogenic, anti-microbial, anti-viral, anti-malarial, anti-apoptotic and/or neuroprotective therapeutic agent including but not limited to tetrandrine, dexamethasone, remdesivir, GS-441524, artemisinin, ART838, an artemisinin derivative, gilteritinib, baricitinib, venetoclax, sorafenib, islatravir, emtricitabine, tenofovir, tenofovir disoproxil fumarate, tenofovir alafenamide, abacavir, didanosine, lamivudine, stavudine, zidovudine, bictegravir, dolutegravir, elvitegravir, raltegravir, ulonivirine, doravirine, lenacapavir, zanamivir, valaciclovir hydrochloride, acyclovir, lamivudine, indinavir sulfate, nelfinavir, nevirapine, efavirenz, rilpivirine, delavirdine, atazanavir, darunavir, lopinavir and cabotegravir, or a combination thereof.

In still another embodiment, the drug delivery system described herein comprises poly(lactic-co-glycolic acid) in about 2% by weight, poly(lactic acid) in about 10% by weight, N-methyl-2-pyrrolidone (NMP) and triethyl citrate (TEC) (9/1 w/w) in about 57% by weight, m-cresol in about 30% by weight, and insulin, an insulin analog, tetrandrine, dexamethasone, remdesivir, GS-441524, artemisinin, ART838, an artemisinin derivative, or gilteritinib, or a combination thereof in about 1% by weight.

### 2) The Solvents

In one embodiment, the solvent of the drug delivery system described herein is selected from N-methyl-2-pyrrolidone (NMP), benzyl benzoate (BB), benzyl alcohol (BA), triethyl citrate (TEC), acetyl triethyl citrate (ATEC), ethyl acetate (EA), and acetyl tributyl citrate (ATBC), or combinations thereof.

In yet another embodiment, the solvent combination of the drug delivery system described herein is NMP and TEC; NMP and ATEC; NMP and ATBC; NMP and BB; NMP and BA; NMP and EA; TEC and BB; ATEC and BB; ATBC and BB; TEC and BA; ATEC and BA; ATBC and BA; TEC and EA; ATEC and EA; ATBC and EA; NMP, TEC and BB; NMP, ATEC and BB; NMP, ATBC and BB; NMP, TEC and BA; NMP, ATEC and BA; NMP, ATBC and BA; NMP, TEC and EA; NMP, ATEC and EA; NMP, ATBC and EA; TEC, BB and EA; ATEC, BB and EA; ATBC, BB and EA; TEC, BA and EA; ATEC, BA and EA; ATBC, BA and EA; NMP and DMSO; BB and DMSO; BA and DMSO; TEC and DMSO; ATEC and DMSO; ATBC and DMSO; EA and DMSO; NMP, TEC and DMSO; NMP, ATEC and DMSO; NMP, ATBC and DMSO; NMP, BB and DMSO; NMP, BA and DMSO; NMP, EA and DMSO; TEC, ATEC and DMSO; TEC, BB and DMSO; TEC, BA and DMSO; TEC, EA and DMSO; ATEC, BB and DMSO; ATEC, BA and DMSO; ATEC, EA and DMSO; BB, BA and DMSO; BB, EA and DMSO; or BA, EA and DMSO.

In still another embodiment, the solvent combination of the drug delivery system described herein is NMP and TEC, NMP and ATEC, NMP and BB, NMP and BA, NMP and EA or DMSO and TEC.

In other embodiments, the solvent combination of the drug delivery system described herein is NMP and TEC, NMP and ATEC, NMP and BB, or NMP and BA.

### 3) The Alkylphenols

Alkylphenols are a family of organic compounds obtained by the alkylation of phenols. The term is usually reserved for commercially important propylphenol, butylphenol, amylphenol, heptylphenol, octylphenol, nonylphenol, dodecylphenol and related “long chain alkylphenols” (LCAPs). Methylphenols and ethylphenols are also alkylphenols, but they are more commonly referred to by their specific names, cresols and xylenols.

Cresols (also known as hydroxytoluene, toluenol, benzol or cresylic acid) are a group of aromatic organic compounds. They are widely-occurring phenols (sometimes called phenolics) which may be either natural or manufactured. They are also categorized as methylphenols. There are three forms (isomers) of cresol: ortho-cresol (o-cresol), meta-cresol (m-cresol), and para-cresol (p-cresol). These forms occur separately or as a mixture, which can also be called cresol or more specifically, tricresol.

Surprisingly, the studies described herein demonstrate that the subject nanogel formulations containing m-cresol resulted in superior stability and release of the therapeutic agents, specifically insulin and tetrandrine.

### 4) The Therapeutic Agent (Aka, the Active Pharmaceutical Ingredient)

Any therapeutic agent known to those of ordinary skill in the art to be of benefit in the diagnosis, treatment or prevention of a disease is contemplated as a therapeutic agent in the context of the present invention. Therapeutic agents include pharmaceutically active compounds, hormones, growth factors, enzymes, DNA, plasmid DNA, RNA, siRNA, viruses, proteins, lipids, pro-inflammatory molecules, antibodies, antibiotics, anti-inflammatory agents, anti-sense nucleotides and transforming nucleic acids, chemotherapeutics, or combinations thereof. Other non-limiting examples include anti-thrombogenic agents; antioxidants; angiogenic and anti-angiogenic agents and factors; anti-proliferative agents (e.g., agents capable of blocking smooth muscle cell proliferation); anti-inflammatory agents; calcium entry blockers; antineoplastic/antiproliferative/anti-mitotic agents; antimicrobials; antivirals; anti-cancer agents; anti-apoptotic agents, antifungals; anesthetic agents; anti-coagulants; vascular cell growth promoters; vascular cell growth inhibitors; cholesterol-lowering agents; vasodilating agents; agents which interfere with endogenous vasoactive mechanisms; and survival genes which protect against cell death Any of the therapeutic agents may be combined to the extent such combination is biologically compatible. Small molecule and hydrophobic therapeutic agents are preferred in some embodiments.

The therapeutic agents are but not limited to insulin, an insulin analog, tetrandrine, dexamethasone, remdesivir, GS-441524, artemisinin, ART838, an artemisinin derivative, gilteritinib, baricitinib, venetoclax, sorafenib, islatravir, emtricitabine, tenofovir, tenofovir disoproxil fumarate, tenofovir alafenamide, abacavir, didanosine, lamivudine, stavudine, zidovudine, bictegravir, dolutegravir, elvitegravir, raltegravir, ulonivirine, doravirine, lenacapavir, zanamivir, valaciclovir hydrochloride, acyclovir, lamivudine, indinavir sulfate, nelfinavir, nevirapine, efavirenz, rilpivirine, delavirdine, atazanavir, darunavir, lopinavir and cabotegravir, or a combination thereof.

With the primary components of the subject polymer matrix drug delivery system having been described in detail, the various embodiments thereof will be described.

In some embodiments, the drug delivery system of the subject invention is an injectable polymer matrix drug delivery system comprising, a) a biodegradable polymer selected from the group consisting of polyester, poly(lactic-co-glycolic acid) (PLGA), poly(lactic acid), poly(ε-caprolactone), poly(ethylene glycol-block-lactic acid), poly(alkylcyanoacrylate), polyanhydride, poly(bis(p-carboxyphenoxy) propane-sebacic acid), polyorthoester, polyphosphoester, polyphosphazene, polyurethane, and poly(amino acid), or combinations thereof, either among themselves or their copolymers and/or blends with poly(ethylene glycol) (PEG).
b) a solvent or a combination of solvents;
c) an alkylphenol; and
d) insulin, an insulin analog, tetrandrine, dexamethasone, remdesivir, GS-441524, artemisinin, ART838, an artemisinin derivative, or gilteritinib, or a combination thereof.

In an embodiment, d) is insulin. In another embodiment, the alkylphenol is a cresol. In yet another embodiment, the alkylphenol is m-cresol.

In a preferred embodiment, the drug delivery system described herein is an injectable polymer matrix drug delivery system comprising:
a) poly(lactic-co-glycolic acid) (PLGA) and poly(lactic acid) (PLA);
b) N-methyl-2-pyrrolidone (NMP) and triethyl citrate (TEC);
c) m-cresol; and
d) insulin, an insulin analog, tetrandrine, dexamethasone, remdesivir, GS-441524, artemisinin, ART838, an artemisinin derivative, or gilteritinib, or a combination thereof.

In another preferred embodiment, the drug delivery system described herein is an injectable polymer matrix drug delivery system comprising:
a) 2% PLGA by weight and 10% PLA by weight;
b) 57% NMP and TEC by weight;
c) 30% m-cresol by weight; and
d) 1% of insulin, an insulin analog, tetrandrine, dexamethasone, remdesivir, GS-441524, artemisinin, ART838, an artemisinin derivative, or gilteritinib, or a combination thereof by weight.

In yet another preferred embodiment, the drug delivery system described herein is an injectable polymer matrix drug delivery system comprising:
a) 6% PLGA by weight and 6% PLA by weight;
b) 57% NMP and TEC by weight;
c) 30% m-cresol by weight; and
d) 1% of insulin, an insulin analog, tetrandrine, dexamethasone, remdesivir, GS-441524, artemisinin, ART838, an artemisinin derivative, or gilteritinib, or a combination thereof by weight.

In one embodiment, the active pharmaceutical ingredient of the drug delivery system described herein is insulin or an insulin analog, tetrandrine, dexamethasone, remdesivir, GS441524, artemisinin, ART838, an artemisinin derivative, or gilteritinib, or a combination thereof.

In another embodiment, the active pharmaceutical ingredient of the drug delivery system described herein is insulin.

In yet another embodiment, the active pharmaceutical ingredient of the drug delivery system described herein is an insulin analog.

In still another embodiment of the drug delivery system described herein, the active pharmaceutical ingredient is insulin or an insulin analog, and the solvent is selected from N-methyl-2-pyrrolidone (NMP), benzyl benzoate (BB), benzyl alcohol (BA), triethyl citrate (TEC), acetyl triethyl citrate (ATEC), and ethyl acetate (EA), or combinations thereof, the biodegradable polymer is selected from poly(L-lactic acid) and poly(D,L-lactic acid), or combinations thereof.

In still another embodiment, the active pharmaceutical ingredient of the drug delivery system described herein is insulin.

In yet another embodiment, active pharmaceutical ingredient of the drug delivery system described herein is an insulin analog.

In another embodiment, the active pharmaceutical ingredient of the drug delivery system described herein is tetrandrine.

In another embodiment, the active pharmaceutical ingredient of the drug delivery system described herein is dexamethasone.

In another embodiment, the active pharmaceutical ingredient of the drug delivery system described herein is remdesivir.

In another embodiment, the active pharmaceutical ingredient of the drug delivery system described herein is GS-441524.

In another embodiment, the active pharmaceutical ingredient of the drug delivery system described herein is artemisinin.

In another embodiment, the active pharmaceutical ingredient of the drug delivery system described herein is ART838.

In another embodiment, the active pharmaceutical ingredient of the drug delivery system described herein is gilteritinib.

In another embodiment, the active pharmaceutical ingredient of the drug delivery system described herein is a combination of remdesivir and dexamethasone.

In another embodiment, the active pharmaceutical ingredient of the drug delivery system described herein is a combination of GS-441524 and dexamethasone. In another embodiment, the active pharmaceutical ingredient of the drug delivery system described herein is a combination of ART838 and gilteritinib.

In still another embodiment of the drug delivery system described herein, the active pharmaceutical ingredient is insulin, an insulin analog, tetrandrine, dexamethasone, remdesivir, GS-441524, artemisinin, ART838, an artemisinin derivative, or gilteritinib, or a combination thereof, and the solvent is selected from N-methyl-2-pyrrolidone (NMP), benzyl benzoate (BB), benzyl alcohol (BA), triethyl citrate (TEC), acetyl triethyl citrate (ATEC), and ethyl acetate (EA), or a combination thereof, the biodegradable polymer is selected from poly(L-lactic acid) and poly(D,L-lactic acid), or a combination thereof.

In an embodiment of the drug delivery system described herein, the system comprises poly(lactic-co-glycolic acid) (PLGA), poly(lactic acid) (PLA), insulin or an insulin analog, N-methyl-2-pyrrolidone (NMP) and triethyl citrate (TEC).

In one embodiment of the drug delivery system described herein, the PLA comprises a first PLA having an inherent viscosity of about 0.40-0.70 dL/g and a second optional PLA having an inherent viscosity of about 0.40-0.70 dL/g that is different than the inherent viscosity of the first PLA. In another embodiment, the inherent viscosity of the first PLA is about 0.63 dL/g. In another embodiment, the inherent viscosity of the second PLA is about 0.47 dL/g.

In one embodiment of the drug delivery system described herein, the PLGA is comprised of approximately 50% lactic acid and approximately 50% glycolic acid.

In one embodiment of the drug delivery system described herein, the PLGA is comprised of approximately 50% lactic acid and approximately 50% glycolic acid.

In one embodiment of the drug delivery system described herein, the NMP and TEC are in a ratio of approximately 9:1, respectively.

In one embodiment of the drug delivery system described herein, the system is comprised of approximately 1-10% PLGA by weight, approximately 1-20% PLA by weight, 1-50% m-cresol, 1-10% by weight insulin, an insulin analog, tetrandrine, dexamethasone, remdesivir, GS-441524, artemisinin, ART838, an artemisinin derivative, gilteritinib, or a combination thereof, and 50-80% NMP and TEC by weight.

In one embodiment of the drug delivery system described herein, the system is comprised of approximately 2% PLGA by weight, approximately 1-10% PLA by weight, 20-40% m-cresol, 1-4% by weight insulin, an insulin analog, tetrandrine, dexamethasone, remdesivir, GS-441524, artemisinin, ART838, an artemisinin derivative, gilteritinib, or a combination thereof, and 50-60% NMP and TEC by weight.

In a preferred embodiment of the drug delivery system described herein, the system is comprised of approximately 2% PLGA by weight, approximately 10% PLA by weight, 30% m-cresol, 1% by weight insulin, an insulin analog, tetrandrine, dexamethasone, remdesivir, GS-441524, artemisinin, ART838, an artemisinin derivative, gilteritinib, or a combination thereof, and 57% NMP and TEC by weight.

In another preferred embodiment of the drug delivery system described herein, the system is comprised of approximately 6% PLGA by weight, approximately 6% PLA by weight, 30% m-cresol, 1% by weight insulin, an insulin analog, tetrandrine, dexamethasone, remdesivir, GS-441524, artemisinin, ART838, an artemisinin derivative, gilteritinib, or a combination thereof, and 57% NMP and TEC by weight.

In another embodiment of the drug delivery system described herein, the system comprises poly(lactic-co-glycolic acid) (PLGA), poly(lactic acid) (PLA), insulin, an insulin analog, tetrandrine, dexamethasone, remdesivir, GS-441524, artemisinin, ART838, an artemisinin derivative, gilteritinib, or a combination thereof, N-methyl-2-pyrrolidone (NMP) and ethyl acetate (EA).

In one embodiment of the drug delivery system described herein, the PLA comprises a first PLA having an inherent viscosity of about 0.40-0.70 dL/g and a second PLA having an inherent viscosity of about 0.40-0.70 dL/g that is different than the inherent viscosity of the first PLA. In another embodiment, the inherent viscosity of the first PLA is about 0.63 dL/g. In another embodiment, the inherent viscosity of the second PLA is about 0.47 dL/g. In one embodiment of the drug delivery system described herein, the PLGA is comprised of approximately 50% lactic acid and approximately 50% glycolic acid.

In one embodiment of the drug delivery system described herein, the PLGA is comprised of approximately 50% lactic acid and approximately 50% glycolic acid.

In one embodiment of the drug delivery system described herein, the NMP and TEC are in a ratio of approximately 9:1, respectively.

In one embodiment of the drug delivery system described herein, the system is comprised of approximately 1-10% PLGA by weight, approximately 1-20% PLA by weight, 1-50% m-cresol, 1-10% by weight insulin, an insulin analog, tetrandrine, dexamethasone, remdesivir, GS-441524, artemisinin, ART838, an artemisinin derivative, or gilteritinib, or a combination thereof, and 55-88% NMP and EA by weight.

In one embodiment of the drug delivery system described herein, the system is comprised of approximately 1-4% PLGA by weight, approximately 1-10% PLA by weight, 20-40% m-cresol, 1-5% by weight insulin, an insulin analog, tetrandrine, dexamethasone, remdesivir, GS-441524, artemisinin, ART838, an artemisinin derivative, or gilteritinib, or a combination thereof, and 72-73.5% NMP and EA by weight.

In one embodiment of the drug delivery system described herein, the system is comprised of approximately 2% PLGA by weight, approximately 10% PLA by weight, 30% m-cresol, 1% by weight insulin, an insulin analog, tetrandrine, dexamethasone, remdesivir, GS-441524, artemisinin, ART838, an artemisinin derivative, gilteritinib, or a combination thereof, and 57% NMP and EA by weight.

### B. Methods of Preparation and Use

In another aspect of the invention, provided herein is a method of forming a polymer matrix drug delivery system described herein comprising:
a) adding a therapeutic agent or a combination thereof to an alkylphenol and a solvent or a combination of solvents in a first syringe;
b) adding a biodegradable polymer selected from the group consisting of poly(lactic-co-glycolic acid), poly(lactic acid), poly(ε-caprolactone), poly(ethylene glycol-block-lactic acid), poly(alkylcyanoacrylate), polyanhydride, poly(bis(pcarboxyphenoxy) propane-sebacic acid), polyorthoester, polyphosphoester, polyphosphazene, polyurethane, and poly(amino acid), or combinations thereof to a second syringe;
c) sterilizing the two syringes by gamma irradiation; and
d) mixing the components of the two syringes at the time of injection.

In another embodiment, a method of forming a polymer matrix drug delivery system comprises the steps of: a) adding a therapeutic agent or a combination thereof to an alkylphenol and a solvent or a combination of solvents to form a composition; and b) adding a biodegradable polymer selected from the group consisting of poly(lactic-coglycolic acid), poly(lactic acid), poly(ε-caprolactone), poly(ethylene glycol-block-lactic acid), poly(alkylcyanoacrylate), polyanhydride, poly(bis(p-carboxyphenoxy) propanesebacic acid), polyorthoester, polyphosphoester, polyphosphazene, polyurethane, and poly(amino acid), or combinations thereof to the composition.

In an embodiment, the therapeutic agent in either or both of the two above-described methods is insulin or an insulin analog.

In an embodiment, the alkylphenol in any or all of the foregoing methods is a cresol. In another embodiment, in any or all of the foregoing methods the alkylphenol is m-cresol.

In yet another embodiment, the biodegradable polymer in any or all of the foregoing methods is a combination of poly(lactic-co-glycolic acid) and poly(lactic acid).

In still another embodiment, the solvent in any or all of the foregoing methods is a combination of NMP and TEC.

In a preferred embodiment, the alkylphenol in any or all of the foregoing methods is m-cresol, the solvent is a combination of NMP and TEC, and the biodegradable polymer is a combination of poly(lactic-co-glycolic acid) and poly(lactic acid).

The components of the two syringes are physically mixed at the time of administration to the patient as follows: one syringe containing the polymer solution is injected into the second syringe containing the drug solution and then injected into the subject.

### C. Product by Process

In an aspect, provided herein is a polymer matrix drug delivery system prepared by the methods described supra.

### D. Methods of Treatment

Provided herein are methods of treatment related to administration of the injectable drug delivery system described herein. Any disease or condition that can benefit from longer term administration of a biologic or drug is suitable for treatment using the invention described herein. For example, common diseases and conditions that are treated using biologics include, but are not limited to type 1 and 2 diabetes, inflammation, hearing loss, COVID-19, HIV, viral infection, cancer, in particular leukemia, and in more particular acute myeloid leukemia, malaria, melanoma, glioblastoma, rheumatoid arthritis, injury such as fractures, Non-Hodgkin lymphoma, carcinoma/adenocarcinoma, basal cell carcinoma, squamous cell carcinoma and transitional cell carcinoma, sarcoma, lymphoma, myeloma, brain and spinal cord cancers, and the like and needed regeneration of tissues. The invention can be used in treating any tissue or organ of the body, including but not limited to neurological tissue, eye (including retina), brain, ear, temporomandibular joint, dental disease or injury, oral tissues, facial tissues, blood, bone tissue, cartilage and joint, heart and vascular system, lung, bronchus, skin, muscle, reproductive organs, liver, pancreas, gastrointestinal tract, endocrine tissues or glands, kidney, breast, oral, head, neck, esophageal, thyroid, fat, muscle, gastrointestinal stromal, intrahepatic bile duct, bladder, colon, rectum, vagina, prostate, testicular, pancreas, cervix, uterine, pleura, immune system, and the like, or any disease or condition that would benefit from long-term sustained release of an active agent for treatment. For example, diseases and conditions such as fungal, seizure, stroke, depression, hepatitis C, diabetes, diabetic retinopathy, age-related macular degeneration, glaucoma, dry eye, Alzheimer's disease, Parkinson's disease, neurological disorders, pain, temporomandibular joint disorders, opioid overdose, and the like.

In an embodiment of the methods, provided herein is a method of administering the drug delivery system described herein, wherein upon administration to the subject in need thereof, the active pharmaceutical ingredient is continuously released from about 0 days to about 140 days.

In another embodiment, provided herein is a method of administering the drug delivery system described herein, wherein upon administration to the subject in need thereof, the active pharmaceutical ingredient is released for at least 16 days.

In another embodiment, provided herein is a method of administering the drug delivery system described herein, wherein the polymer matrix is injected through a needle of about 18-gauge to about 26-gauge.

In another embodiment, provided herein is a method of administering the drug delivery system described herein, wherein the polymer matrix is injected through a needle of about 21-gauge.

In another embodiment, provided herein is a method of administering the drug delivery system described herein, wherein the polymer matrix is injected through a needle of about 22-gauge.

In another embodiment, provided herein is a method of administering the drug delivery system described herein, wherein the polymer matrix is injected through a needle of about 23-gauge to about 26-gauge.

In another embodiment, provided herein is a method of administering the drug delivery system described herein, wherein the polymer matrix is injected through a needle of about 23-gauge.

In another embodiment, provided herein is a method of administering the drug delivery system described herein, wherein the system is formulated for subcutaneous injection or intramuscular injection.

In another embodiment, provided herein is a method of administering the drug delivery system described herein, wherein the system forms a semi-solid or solid depot at the injection site.

### Treatment of Diabetes Mellitus

Type 1 diabetes is characterized by high blood glucose levels caused by little or no insulin secretion from the pancreatic beta cells because of the complete destruction of beta cells by the body's immune system. The World Health Organization (WHO) has estimated that about 422 million people worldwide had diabetes mellitus in 2014. According to the American Diabetes Association, 1.25 million people in United States have type 1 diabetes and the number of newly diagnosed people each year will be approximately 40,000. As the pancreas no longer produces insulin in type 1 diabetes, insulin replacement therapy has been the mainstay of treatment for type 1 diabetes. In healthy individuals, insulin is continuously secreted from pancreas at a rate of 0.5-1 Unit/h throughout the day to maintain the basal insulin level necessary to maintain blood glucose level. In type 1 diabetes, injectable so-called “long-acting” insulin analogs insulin glargine and “ultra-long-acting” insulin analogs such as insulin degludec are usually prescribed to maintain basal insulin level for only a 24-hour period. Owing to their short half-life, these insulin analogues require daily subcutaneous (Sub-Q) injection to maintain basal insulin requirements. This invasive and painful nature of insulin delivery presents safety issues as well as medication non-adherence among patients.

Accordingly, there is an unmet need for a delivery system that can deliver insulin for longer than a 24-hour duration after a single Sub-Q injection to address the safety issues and improve patient compliance in the diabetes market.

In one aspect, provided herein is a method of treating diabetes mellitus, the method comprising administering to a subject in need thereof the injectable polymer matrix drug delivery system described herein. In an embodiment, the diabetes mellitus is type 1 diabetes. In another embodiment, the diabetes mellitus is type 2 diabetes.

In an embodiment, the method of treating diabetes comprises administering to the subject in need thereof the injectable polymer matrix drug delivery system comprising: a) poly(lactic-co-glycolic acid) and poly(lactic acid); b) N-methyl-2-pyrrolidone (NMP) and triethyl citrate (TEC); c) m-cresol; and d) an insulin or an insulin analog.

In another embodiment, the method of treating diabetes comprises administering to the subject in need thereof the injectable polymer matrix drug delivery system comprising poly(lactic-co-glycolic acid) in about 2% by weight, poly(lactic acid) in about 10% by weight, N-methyl-2-pyrrolidone (NMP) and triethyl citrate (TEC) (9/1 w/w) in about 57% by weight, m-cresol in about 30% by weight, and insulin or an insulin analog in about 1% by weight.

In another embodiment, the method of treating diabetes comprises administering to the subject in need thereof the injectable polymer matrix drug delivery system comprising poly(lactic-co-glycolic acid) in about 6% by weight, poly(lactic acid) in about 6% by weight, N-methyl-2-pyrrolidone (NMP) and triethyl citrate (TEC) (9/1 w/w) in about 57% by weight, m-cresol in about 30% by weight, and insulin or an insulin analog in about 1% by weight.

### E. Examples

This invention is not limited to the particular processes, compositions, or methodologies described, as these may vary. The terminology used in the description is for the purpose of describing the particular versions or embodiments only and is not intended to limit the scope of the present invention which will be limited only by the appended claims. Although any methods and materials similar or equivalent to those described herein can be used in the practice or testing of embodiments of the present invention, the preferred methods, devices, and materials are now described. All publications mentioned herein are incorporated by reference in their entirety; nothing herein is to be construed as an admission that the invention is not entitled to antedate such disclosure by virtue of prior invention.

### Example 1

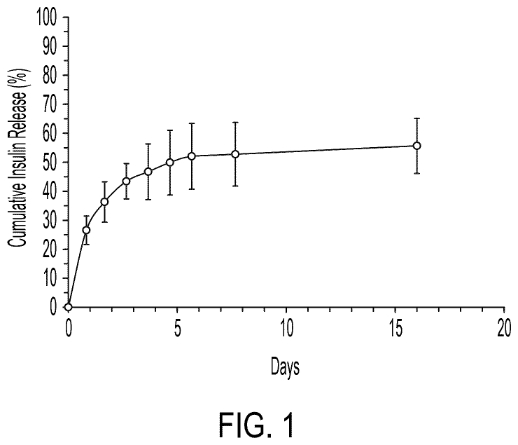

Table 1 (see below) provides the component weight percentages for two example formulations as described herein. Upon injection of these formulations in a subject in need thereof, a drug depot forms in the subcutaneous tissue of the subject. This depot allows for sustained release of insulin (the active therapeutic agent). It was found that insulin release continued to occur for greater than 16 days after injection of formulation F-1 (FIG. 1).

TABLE 1

| | PLGA | PLA | PLA | | | NMP/TEC |
|---|---|---|---|---|---|---|
| | (wt %) | (wt %) | (wt %) | Insulin | m-Cresol | 9:1 |
| Formulation | i.v.-0.63 | i.v.-0.63 | i.v.-0.47 | (wt %) | (wt %) | (wt %) |
| F-1 | 2 | 2 | 8 | 1 | 30 | 57 |
| F-2 | 2 | 2 | 8 | 1 | 30 | 57 |
| F-3 | 2 | 2 | 8 | 1 | | 87 |
| F-4 | 2 | 2 | 8 | 4 | 30 | 54 |
| F-5 | 2 | 10 | — | 1 | 30 | 57 |
| F-6 | 3 | 3 | 12 | 1 | 30 | 51 |
| F-7 | 2 | 2 | 8 | 3 | 30 | 55 |
| F-8 | 6 | 6 | | 1 | 30 | 57 |
| F-12 | 4 | 6 | 2 | 1 | 30 | 57 |
| F-13 | 2 | 6 | 4 | 1 | 30 | 57 |
| F-14 | 2 | 4 | 6 | 1 | 30 | 57 |
| F-15 | 4 | 4 | 4 | 1 | 30 | 57 |
| F-16 | 3 | 3 | 6 | 1 | 30 | 57 |

### Example 2. m-Cresol Provides Insulin Stability and Sustained Release

The release of insulin with the formulations described herein were monitored in the presence (F-1 and F-2) and absence (F-3) of m-cresol. The results are summarized in Table 2.

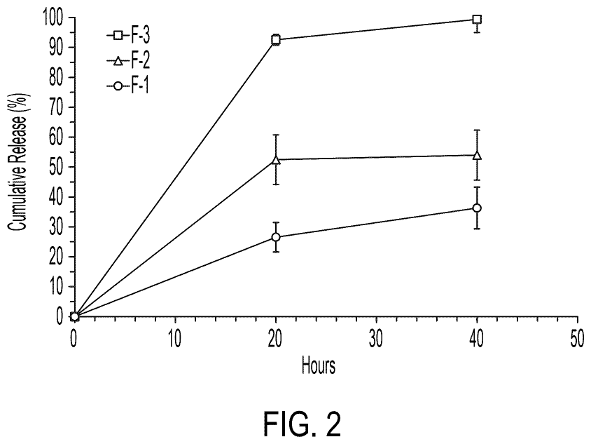

Surprisingly, formulations containing m-cresol (F-1 and F-2) resulted in superior insulin stability and release. After twenty hours, nearly all of the insulin in F-3 had been released, whereas F-1 and F-2 exhibited a much slower release profile (FIG. 2). These results indicate that the addition of m-cresol plays a vital role in the stability and release profile of the insulin formulations described herein.

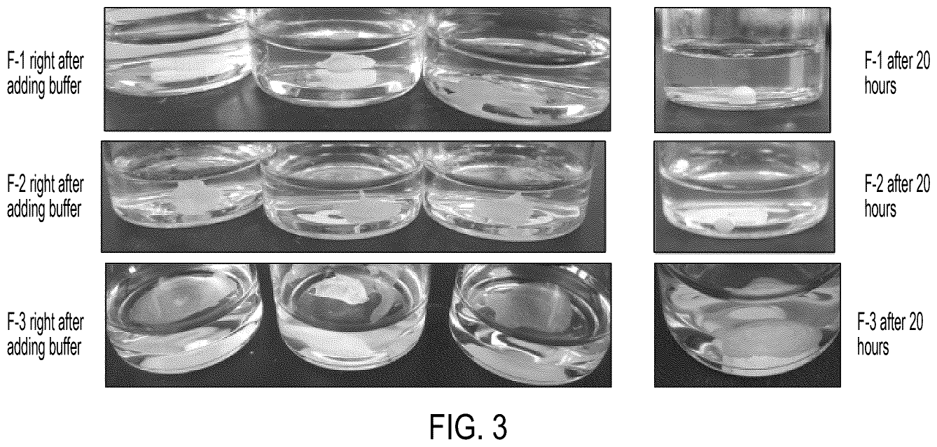

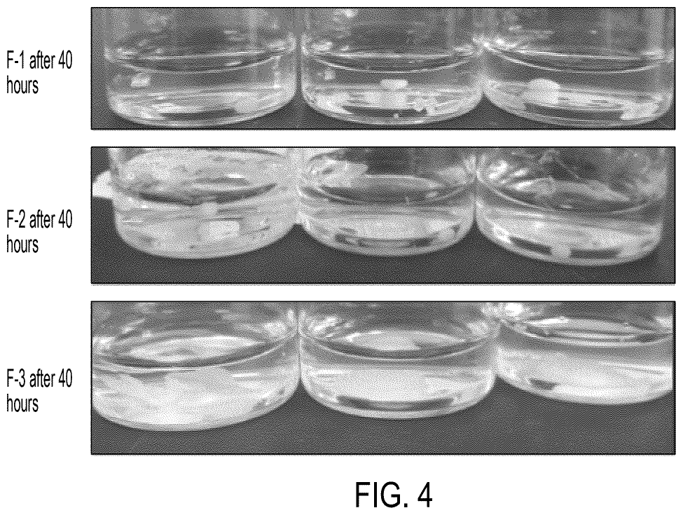

The order of addition is also important for the desired sustained release profile. In F-1, m-cresol and insulin were combined prior to addition of PLGA and PLA, whereas the components of F-2 were added sequentially. After forty hours, only about one third of the insulin of F-1 was released compared to about half the insulin in F-2. These results show that for optimal sustained release of insulin, m-cresol and insulin should be combined first, then added to the rest of the drug delivery components prior to injection. See Table 2, FIG. 3, and FIG. 4.

TABLE 2

| | F-1 | F-2 | F-3 |
|---|---|---|---|
| Time | ½ 96r + | ½ 96r + | ½ 96r + |
| (hour) | (30% m + 1% INS) | (30% m + 1% INS) | (1% INS) |
| 20 | 26.6 | 52.5 | 92.5 |
| 40 | 36.3 | 53.9 | 66.4 |

m = m-cresol;
INS = insulin

Accordingly, in another embodiment, provided herein is a method of administering the drug delivery system described herein, wherein the drug delivery system comprises a formulation selected from any one of tables 1 and 2.

In one embodiment, provided herein is a method of treating diabetes mellitus, the method comprising administering to a subject in need thereof the injectable polymer matrix drug delivery system comprising poly(lactic-co-glycolic acid) (PLGA), poly(lactic acid) (PLA), insulin or an insulin analog, N-methyl-2-pyrrolidone (NMP) and triethyl citrate (TEC).

In one embodiment of the methods described herein, the PLA comprises a first PLA having an inherent viscosity of about 0.40-0.70 dL/g and a second optional PLA having an inherent viscosity of about 0.40-0.70 dL/g that is different than the inherent viscosity of the first PLA. In another embodiment, the inherent viscosity of the first PLA is about 0.63 dL/g. In another embodiment, the inherent viscosity of the second PLA is about 0.47 dL/g.

In one embodiment of the methods described herein, the PLGA is comprised of approximately 50% lactic acid and approximately 50% glycolic acid.

In one embodiment of the methods described herein, the PLGA is comprised of approximately 50% lactic acid and approximately 50% glycolic acid.

In one embodiment of the methods described herein, the NMP and TEC are in a ratio of approximately 9:1, respectively.

In one embodiment of the methods described herein, the system is comprised of approximately 1-10% PLGA by weight, approximately 1-25% PLA by weight, 20-60% m-cresol by weight, 0.1-10% by weight insulin or an insulin analog, and 50-80% NMP and TEC by weight.

In one embodiment of the methods described herein, the system is comprised of approximately 1-10% PLGA by weight, approximately 1-20% PLA by weight, 0.1-5% by weight insulin or an insulin analog, 20-40% m-cresol by weight and 50-60% NMP and TEC by weight.

In one embodiment of the methods described herein, the system is comprised of approximately 2% PLGA by weight, approximately 10% PLA by weight, 30% m609616: cresol by weight 1% by weight insulin or an insulin analog, and 57% NMP and TEC by weight.

In one embodiment of the methods described herein, the system is comprised of approximately 6% PLGA by weight, approximately 6% PLA by weight, 30% m-cresol by weight, 1% by weight insulin or an insulin analog, and 57% NMP and TEC by weight.

In another embodiment, provided herein is a method of reducing blood glucose levels, the method comprising administering to a subject in need thereof the injectable polymer matrix drug delivery system comprising poly(lactic-co-glycolic acid) (PLGA), poly(lactic acid) (PLA), insulin or an insulin analog, N-methyl-2-pyrrolidone (NMP) and ethyl acetate (EA).

In one embodiment of the methods described herein, the PLA comprises a first PLA having an inherent viscosity of about 0.40-0.70 dL/g and a second PLA having an inherent viscosity of about 0.40-0.70 dL/g that is different than the inherent viscosity of the first PLA. In another embodiment, the inherent viscosity of the first PLA is about 0.63 dL/g. In another embodiment, the inherent viscosity of the second PLA is about 0.47 dL/g.

In one embodiment of the methods described herein, the PLGA is comprised of approximately 50% lactic acid and approximately 50% glycolic acid.

In one embodiment of the methods described herein, the NMP and EA are in a ratio of approximately 9:1, respectively.

In one embodiment of the methods described herein, the system is comprised of approximately 1-10% PLGA by weight, approximately 1-25% PLA by weight, 0.1-10% by weight insulin or an insulin analog, and 55-88% NMP and EA by weight.

In one embodiment of the methods described herein, the system is comprised of approximately 1-5% PLGA by weight, approximately 1-20% PLA by weight, 0.1-5% by weight insulin or an insulin analog, and 55-88% NMP and EA by weight.

In one embodiment of the methods described herein, the system is comprised of approximately 2% PLGA by weight, approximately 10% PLA by weight, 1% by weight insulin or an insulin analog, and 72-73.5% NMP and EA by weight.

### Example 3: Management of Blood Glucose Levels

In another aspect, provided herein is a method of reducing blood glucose levels, the method comprising administering to a subject in need thereof the injectable polymer matrix drug delivery system described herein.

In an embodiment, the method of reducing blood glucose levels comprises administering to the subject in need thereof the injectable polymer matrix drug delivery system comprising: a) poly(lactic-co-glycolic acid) and poly(lactic acid); b) N-methyl-2-pyrrolidone (NMP) and triethyl citrate (TEC); c) m-cresol; and d) an insulin or an insulin analog.

In another embodiment, the method of reducing blood glucose levels comprises administering to the subject in need thereof the injectable polymer matrix drug delivery system comprising poly(lactic-co-glycolic acid) in about 2% by weight, poly(lactic acid) in about 10% by weight, N-methyl-2-pyrrolidone (NMP) and triethyl citrate (TEC) (9/1 w/w) in about 57% by weight, m-cresol in about 30% by weight, and insulin or an insulin analog in about 1% by weight.

In another embodiment, the method of reducing blood glucose levels comprises administering to the subject in need thereof the injectable polymer matrix drug delivery system comprising poly(lactic-co-glycolic acid) in about 6% by weight, poly(lactic acid) in about 6% by weight, N-methyl-2-pyrrolidone (NMP) and triethyl citrate (TEC) (9/1 w/w) in about 57% by weight, m-cresol in about 30% by weight, and insulin or an insulin analog in about 1% by weight.

Additional studies were conducted using formulations of the subject drug delivery system as described in Table 3, below, and discussed in the following additional examples.

TABLE 3

| | | % Components | | | | | | | |
|---|---|---|---|---|---|---|---|---|---|
| | | | m- | | PLA | PLA | NMP/TEC | | |
| | Formulation | insulin | cresol | PLGA | (0.63) | (0.47) | (9:1) | Zinc | Chitosan |
| ✓✓ | F1 | 1 | 30 | 50/50-2 | 2 | 8 | 57 | | |
| ✓ | F2 | 1 | 30 | 50/50-4 | 6 | 2 | 57 | | |
| ✓ | F3 | 1 | 30 | 50/50-2 | 6 | 4 | 57 | | |
| ✓ | F4 | 1 | 30 | 50/50-2 | 4 | 6 | 57 | | |
| ✓ | F5 | 1 | 30 | 50/50-4 | 4 | 4 | 57 | | |
| ✓ | F6 | 1 | 30 | 50/50-3 | 3 | 6 | 57 | | |
| ✓ | F7 | 1 | 30 | 50/50-6 | 6 | 0 | 57 | | |
| ✓ | F8 | 1 | 30 | 50/50-4 | 4 | 16 | 45 | | |
| ✓✓ | F9 | 1 | 30 | 85/15-4 | 4 | 16 | 45 | | |
| ✓✓ | F10 | 2 | 30 | 50/50-2 | 2 | 8 | 56 | | |
| ✓✓ | F11 | 1 | 30 | 50/50-1 | 1 | 4 | 63 | | |
| ✓✓ | F12 | 1 | 30 | 85/15-2 | 2 | 8 | 57 | | |
| ✓✓ | F13 | 1 | 30 | 50/50-12 | — | — | 57 | | |
| ✓ | F14 | 1 | 30 | 65/35-12 | — | — | 57 | | |
| ✓ | F15 | 1 | 30 | 75/25-12 | — | — | 57 | | |
| | F16 | 1 | 30 | 50/50-6 | — | — | 63 | | |
| | F17 | 2 | 30 | 50/50-12 | — | — | 56 | | |
| | F18 | 2 | 30 | 50/50-6 | — | — | 62 | | |
| ✓ | F19 | 1 | — | 50/50-2 | 2 | 8 | 87 | | |
| ✓ | F20 | 1 | 30 | 50/50 | — | — | 57 | | |
| | | | | (0.09)-12 | | | | | |
| ✓✓ | F21 | 1 | 30 | 50/50 | — | — | 55.6 | 1.4 | |
| | | | | (0.09)-12 | | | | (2.08 mg) | |
| ✓ | F22 | 1 | 30 | 50/50 | — | — | 56.6 | | 0.4 |
| | | | | (0.09)-12 | | | | | |
| ✓✓ | F23 | 2 | 30 | 50/50-12 | — | — | 53.2 | 2.8 | — |
| | | | | | | | | (4.2 mg) | |
| ✓✓ | F24 | 2 | 30 | 75/25-12 | — | — | 53.2 | 2.8 | — |
| | | | | | | | | (4.2 mg) | |
| ✓✓ | F25 | 2 | 30 | 75/25-2 | 2 | 8 | 53.2 | 2.8 | — |
| | | | | | | | | (4.2 mg) | |
| ✓ | F26 | 1 | 30 | 85/15-4 | 4 | 16 | 43.6 | 1.4 | |

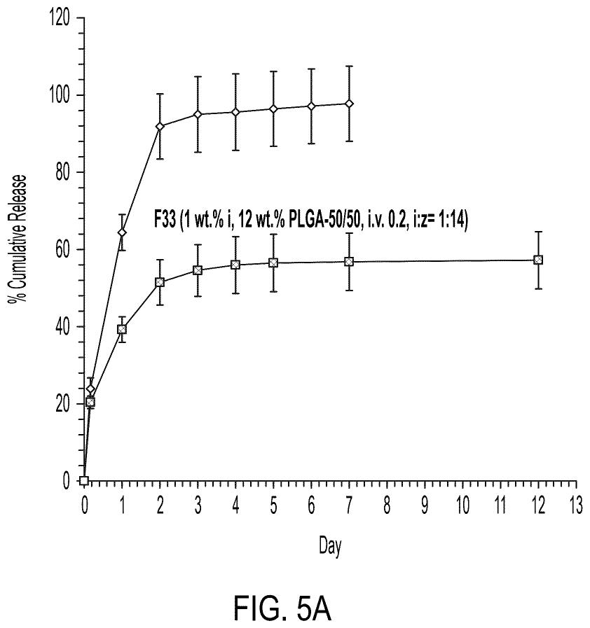

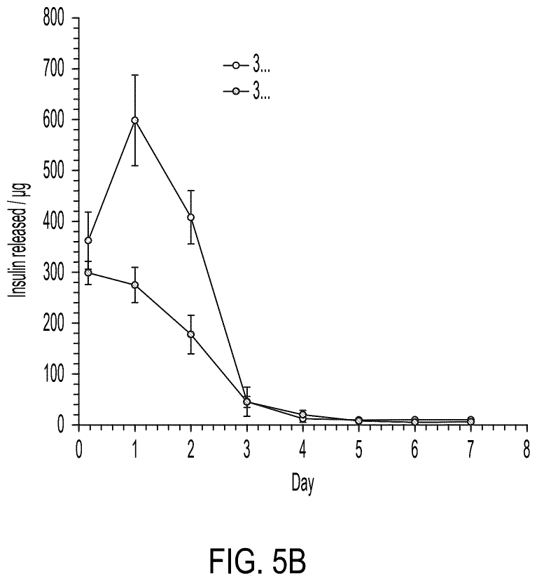

### Example 4: Effect of Insulin Amount In Vitro, i:z=1:14 (FIG. 5A and FIG. 5B)

The effect of insulin amount in vitro, i:z=1:14, was also studied. More specifically, the effect of insulin loading on the cumulative release of insulin from 150 mg ISFGSs containing 12 wt % PLGA 50/50 (i.v. 0.2), 30 wt % meta-cresol, and 1 or 2 wt % insulin and zinc at 1:14 weight ratio was studied. Insulin was released continuously for 5 days from both the formulations and faster and more from the higher insulin loading formulation (2 wt % vs. 1 wt % insulin loading).

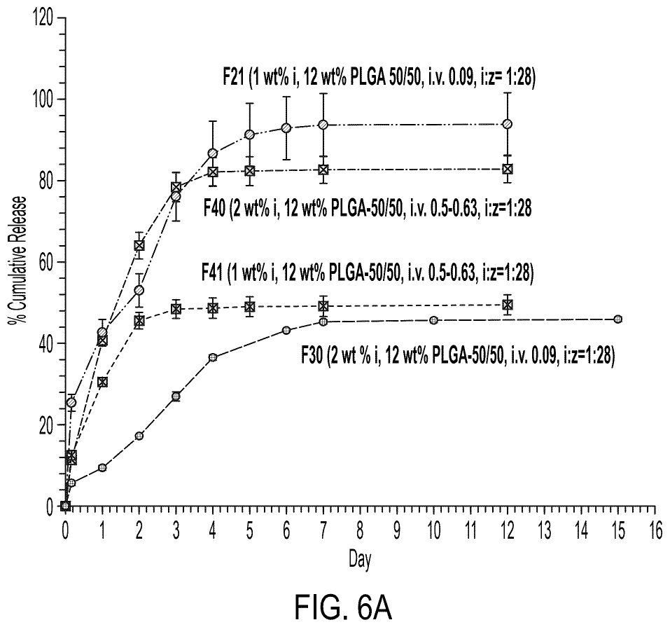

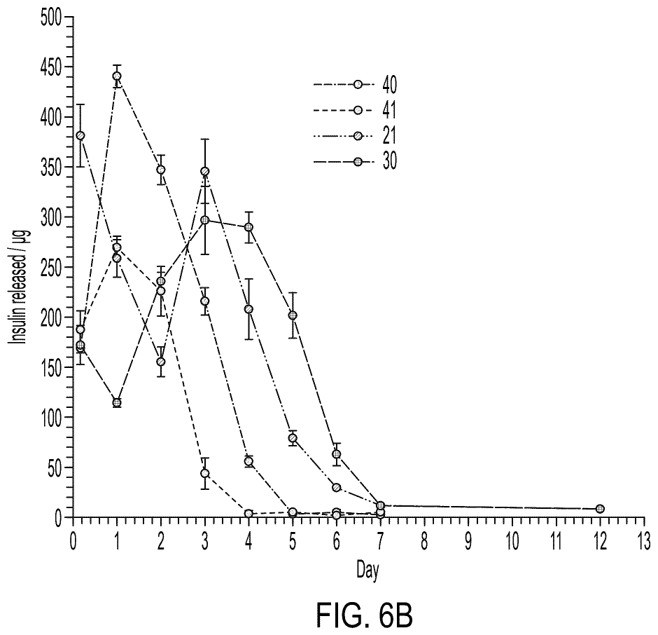

### Example 5: Effect of Insulin Amount and Intrinsic Viscosity In Vitro, i:z=1:28 (FIG. 6A and FIG. 6B)

The effect of insulin amount and intrinsic viscosity in vitro, i:z=1:28, was also studied. More specifically, the effect of insulin loading and polymer intrinsic viscosity on the cumulative release of insulin from 150 mg ISFGSs containing 12 wt % PLGA 50/50 (i.v. 0.09 or 0.5-0.63), 30 wt % meta-cresol, and 1 or 2 wt % insulin and zinc at 1:28 weight ratio was studied. Insulin was released continuously for 4-7 days depending on the formulations and the slowest and longest from the formulation containing higher insulin loading and lower polymer intrinsic viscosity.

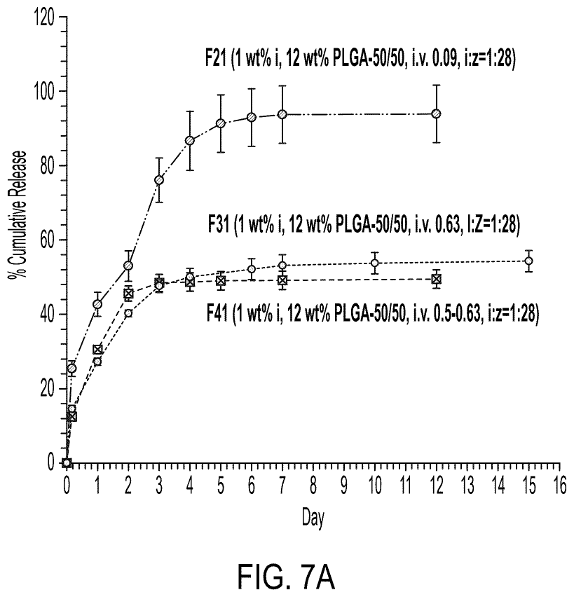

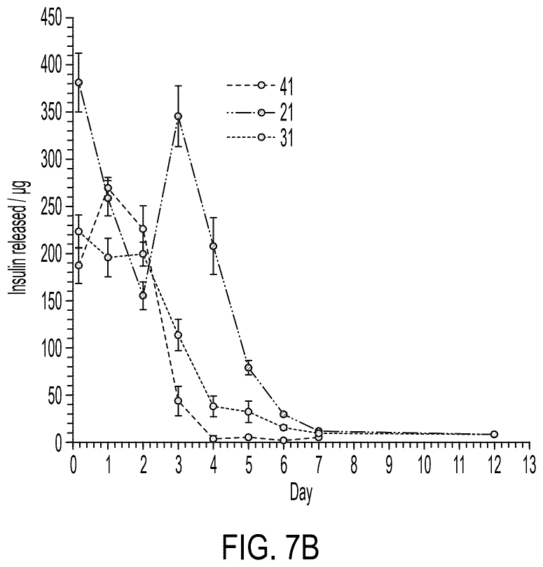

### Example 6: Effect of Intrinsic Viscosity of PLGA In Vitro, 1 wt % Insulin (FIG. 7A and FIG. 7B)

The effect of polymer intrinsic viscosity on the cumulative release of insulin from 150 mg ISFGSs containing 12 wt % PLGA 50/50 (i.v. 0.09, 0.5-0.63, or 0.63), 30 wt % meta-cresol, and 1 wt % insulin and zinc at 1:28 weight ratio was also studied. Insulin was released continuously for 4-7 days depending on the formulations and more and slower from the formulation containing 0.09 intrinsic viscosity.

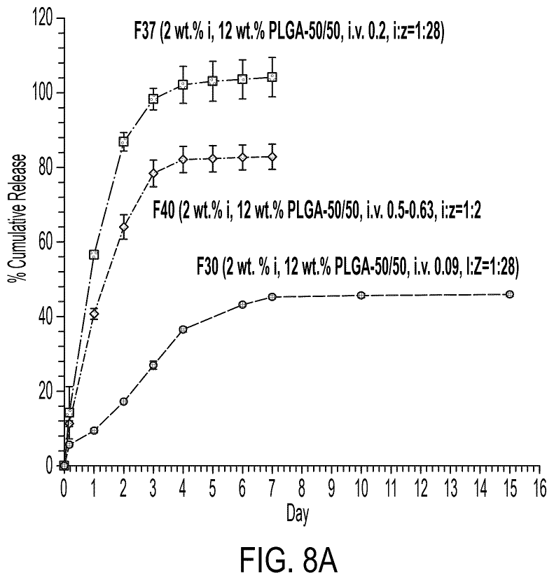

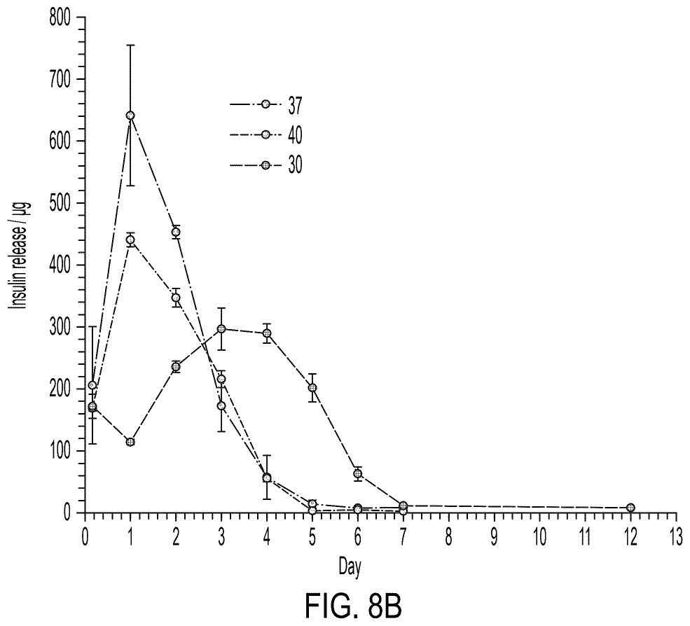

### Example 7: Effect of Intrinsic Viscosity of PLGA In Vitro, 2 wt % Insulin (FIG. 8)

The effect of polymer intrinsic viscosity on the cumulative release of insulin from 150 mg ISFGSs containing 12 wt % PLGA 50/50 (i.v. 0.09, 0.2, or 0.5-0.63), 30 wt % meta-cresol, and 2 wt % insulin and zinc at 1:28 weight ratio was also studied. Insulin was released continuously for 5-7 days depending on the formulations, The slowest and longest release was from the formulation with 0.09 intrinsic viscosity. More insulin was released from formulation containing i.v. 0.2 than 0.5-0.63 during the first 2 days.

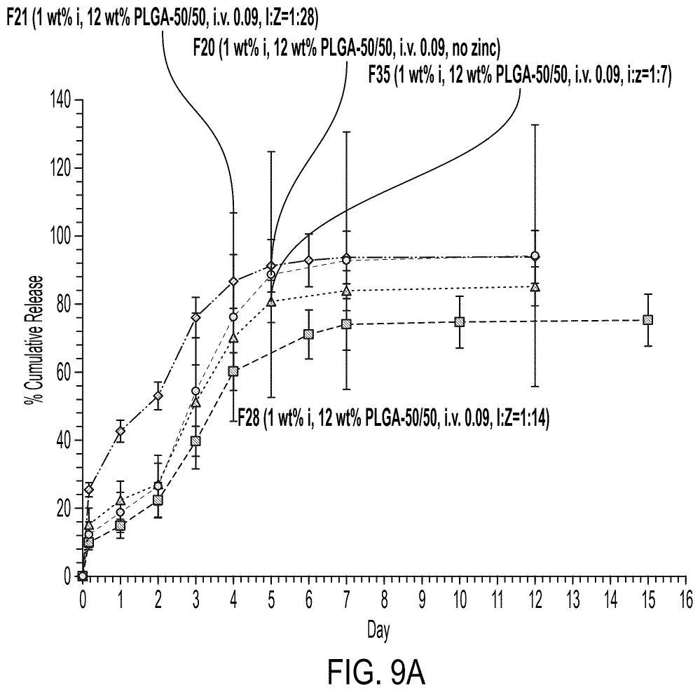

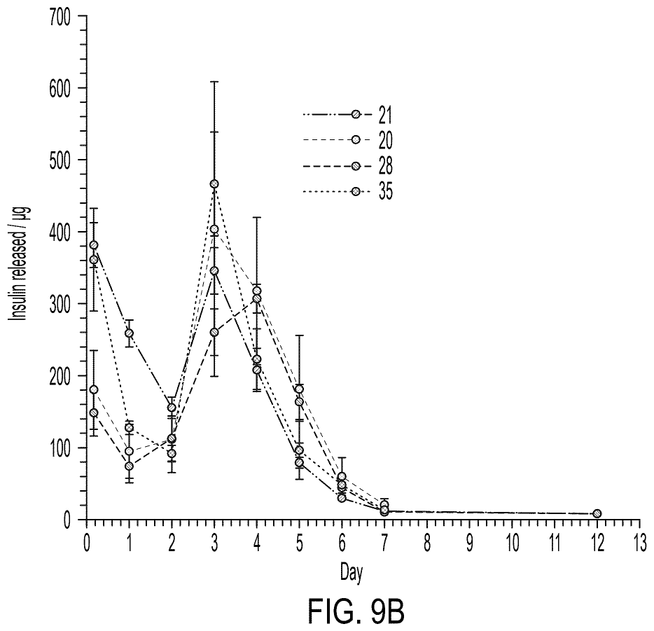

### Example 8: Effect of Zinc Amount In Vitro, 1 wt % Insulin (FIG. 9A and FIG. 9B)

The effect of zinc amount in vitro, 1 wt % insulin was also studied. More specifically, the effect of zinc amount on the cumulative release of insulin from 150 mg ISFGSs containing 12 wt % PLGA 50/50 (i.v. 0.09), 30 wt % meta-cresol, and 1 wt % insulin was studied. Insulin was released continuously for 7 days and the slowest and smoothest (least fluctuation) from the formulation containing insulin:zinc at 1:14 in comparison to 1:7 and 1:28 weight ratios and no zinc.

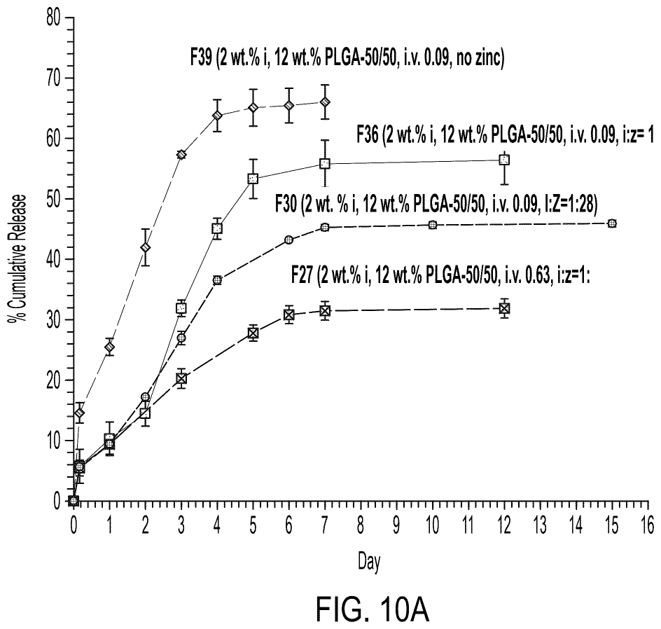

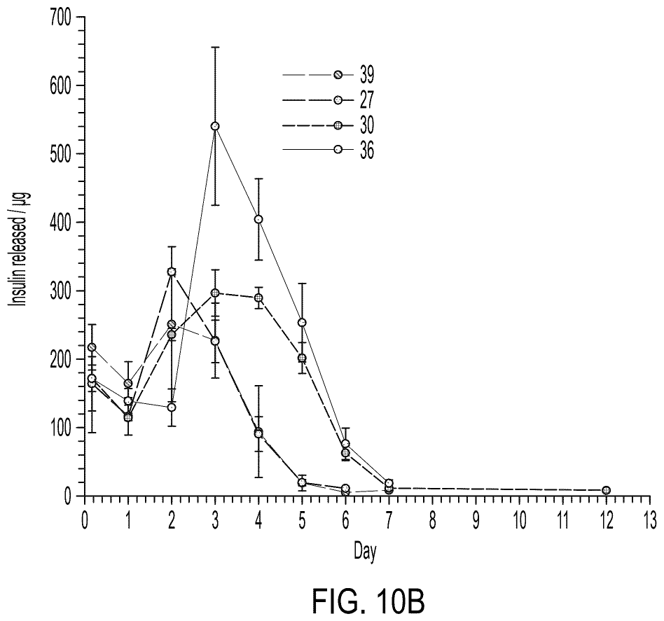

### Example 9: Effect of Zinc Amount In Vitro, 2 wt % Insulin (FIG. 10A and FIG. 10B)

The effect of zinc amount in vitro, 2 wt % insulin was also studied. More specifically, the effect of zinc amount on the cumulative release of insulin from 150 mg ISFGSs containing 12 wt % PLGA 50/50 (i.v. 0.09), 30 wt % meta-cresol, and 1 wt % insulin was studied. Insulin was released continuously for 7 days and the slowest and smoothest (least fluctuation) from the formulation containing insulin:zinc at 1:14 in comparison to 1:7 and 1:28 weight ratios and no zinc.

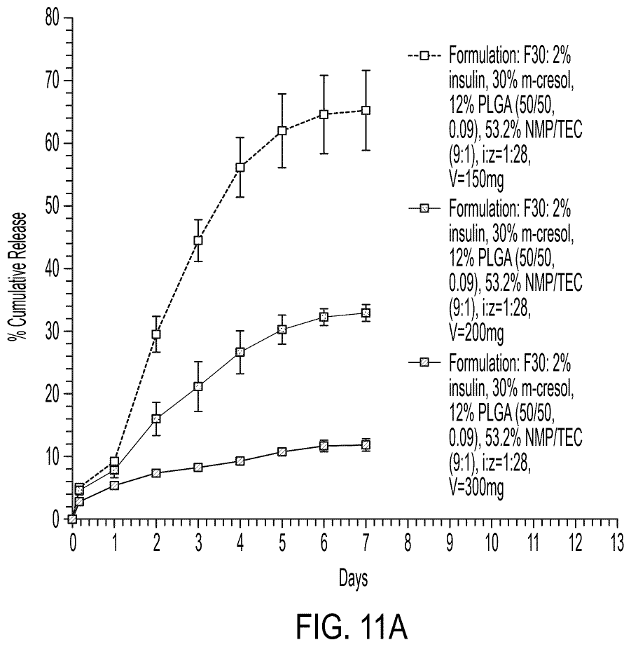

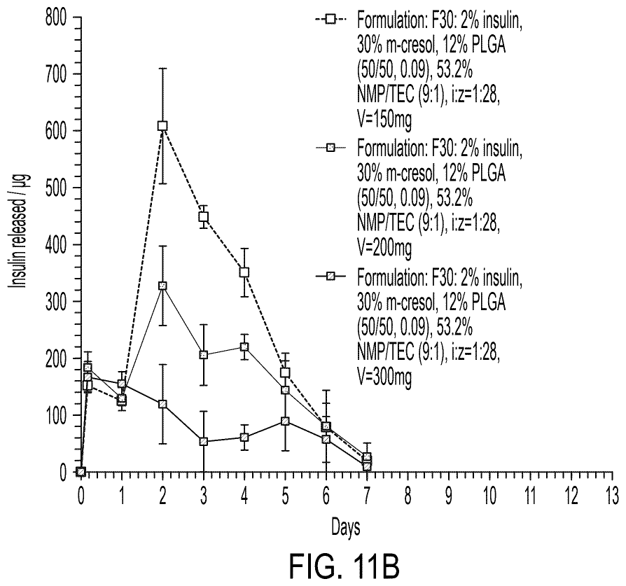

### Example 10: Effect of Formulation Volume In Vitro (FIG. 11A and FIG. 11B)

The effect of formulation volume in vitro was also studied. More specifically, the effect of formulation volume on the cumulative release of insulin from the ISFGSs containing 12 wt % PLGA 50/50 (i.v. 0.09), 30 wt % meta-cresol, 2 wt % insulin, and zinc at insulin:zinc at 1:28 w:w. was studied. Insulin was released continuously for 7 days and slower with increasing the volume of the formulations from 150 to 300 mg.

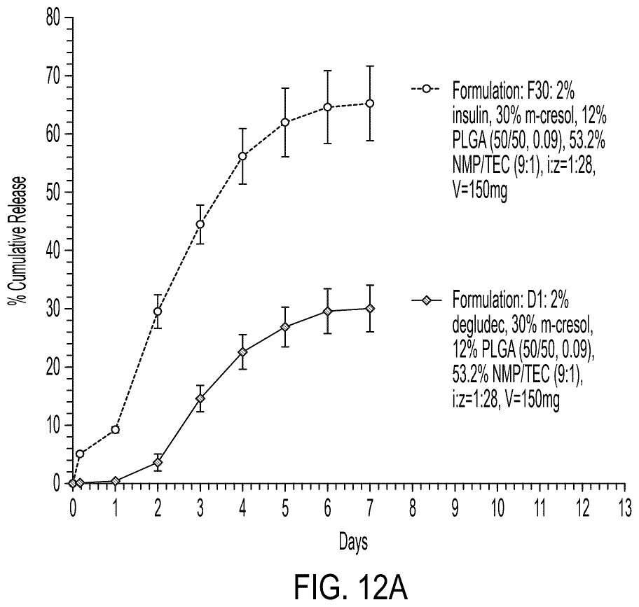

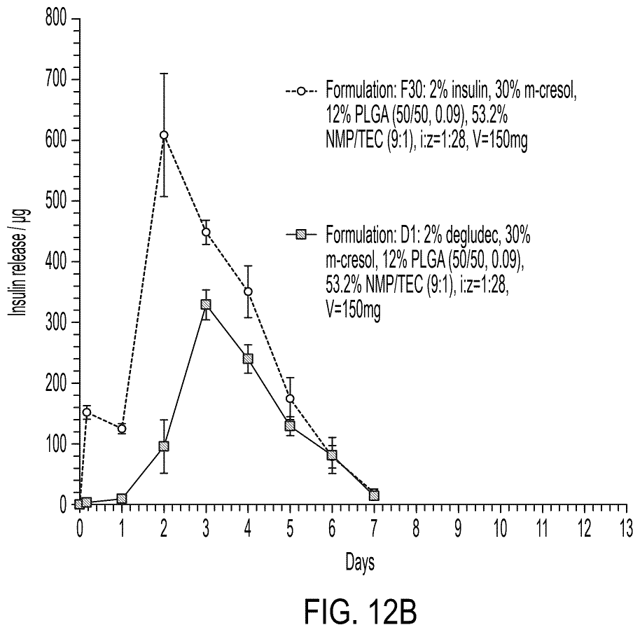

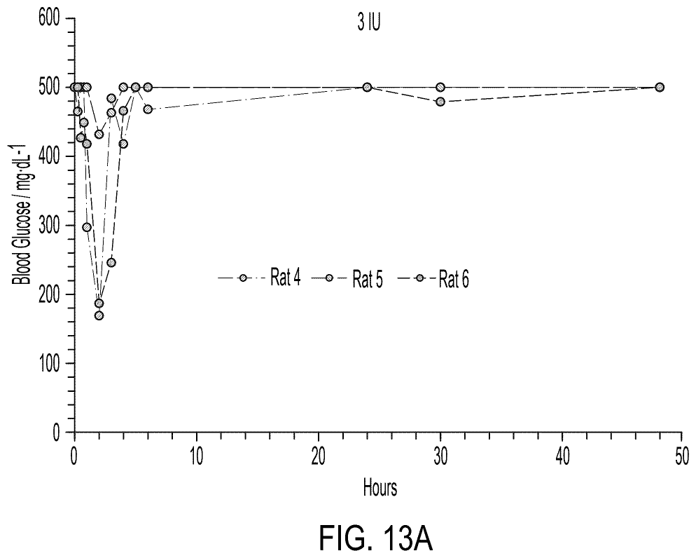

### Example 11: Degludec Vs. Regular Insulin (FIG. 12A and FIG. 12B)

The effect of insulin type on the cumulative release of insulin was also studied. More specifically, the effect of Degludec vs. regular insulin from 150 mg ISFGSs containing 12 wt % PLGA 50/50 (i.v. 0.09), 30 wt % meta-cresol, 2 wt % insulin, and zinc at insulin:zinc at 1:28 w:w. was studied. Insulin was released continuously for 7 days and the slower from the formulation containing Degludec than regular insulin.

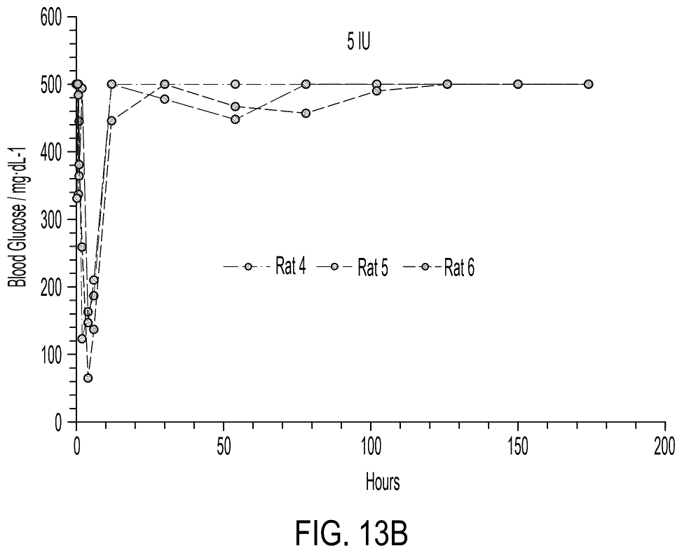

### Example 12: Effect of Sub-Q Injected Insulin (FIG. 13A and FIG. 13B)

The effect of subcutaneously injected insulin as the therapeutic agent component of the subject drug delivery system on diabetic rats was also studied. More specifically, the effect of an insulin depot prepared and delivered in accordance with the teachings of the subject invention on the cumulative release of insulin was studied. Higher insulin dose at 5 IU decreased the blood glucose level to about 100 mg·dL⁻¹. within 4 h after the injection.

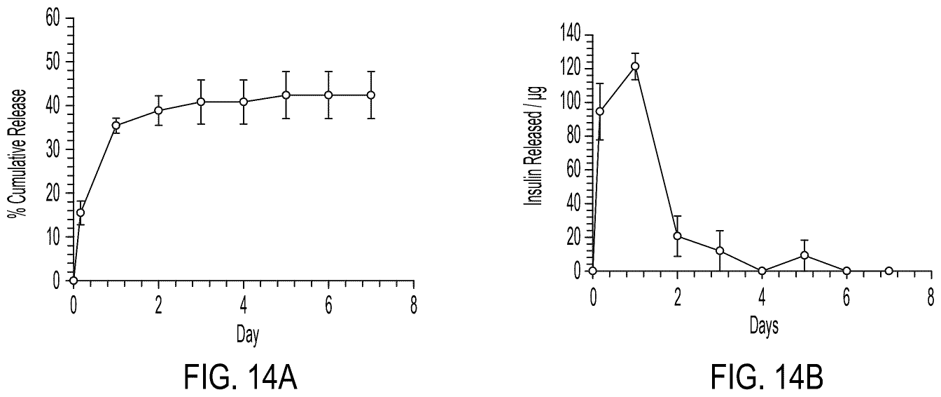

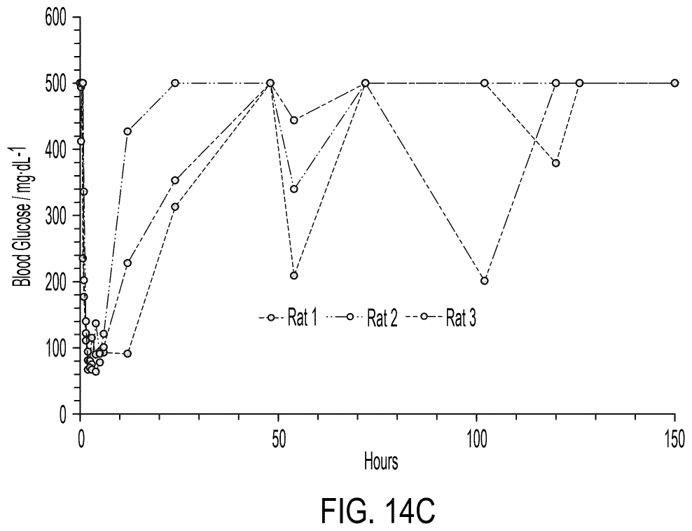

### Example 13: Insulin Release Profile for Formulation F-1 (FIG. 14A, FIG. 14B, and FIG. 14C)

The insulin release of subcutaneously injected insulin using formulation F-1 (Table 3) on diabetic rats was also studied. It was observed that subcutaneously injected 150 mg ISFGSs containing 42 IU insulin decreased the blood glucose level in diabetic rats for 5 days with fluctuation. The ISFGSs were made of 2 wt % PLGA 50/50 (i.v. 0.67), 2 wt % PLA (i.v. 0.63), 8 wt % PLA (i.v. 0.47), 30 wt % meta-cresol, and 1 wt % insulin.

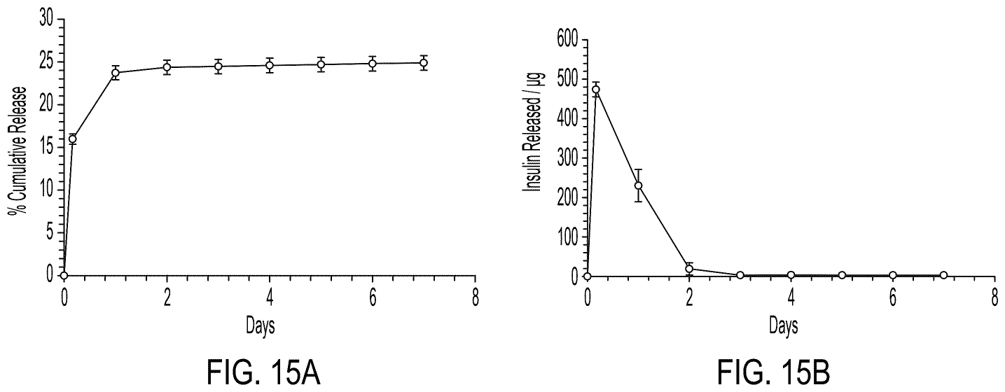

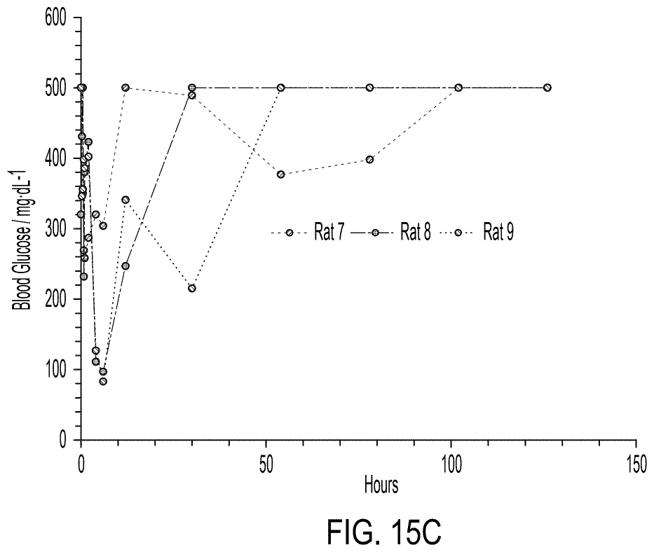

### Example 14: Insulin Release Profile for Formulation F-10 (FIG. 15A, FIG. 15B, and FIG. 15C)

The insulin release of subcutaneously injected insulin using formulation F-10 (Table 3) on diabetic rats was also studied. It was observed that subcutaneously injected 150 mg ISFGSs containing 42 IU insulin decreased the blood glucose level in diabetic rats for 78 hours with fluctuation. The ISFGSs were made of 2 wt % PLGA 50/50 (i.v. 0.67), 2 wt % PLA (i.v. 0.63), 8 wt % PLA (i.v. 0.47), 30 wt % meta-cresol, and 2 wt % insulin.

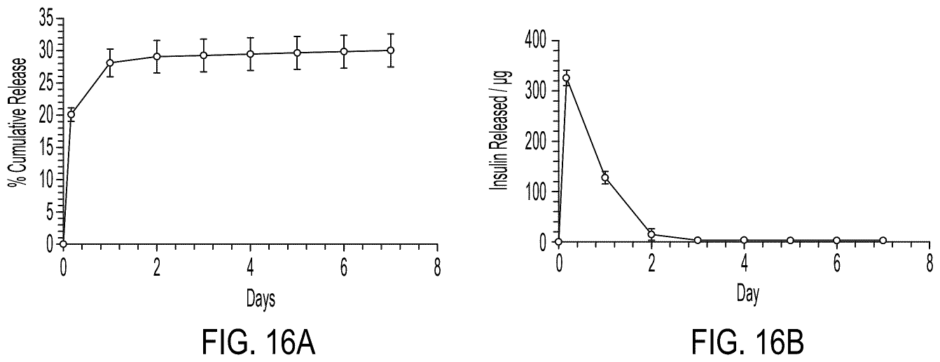

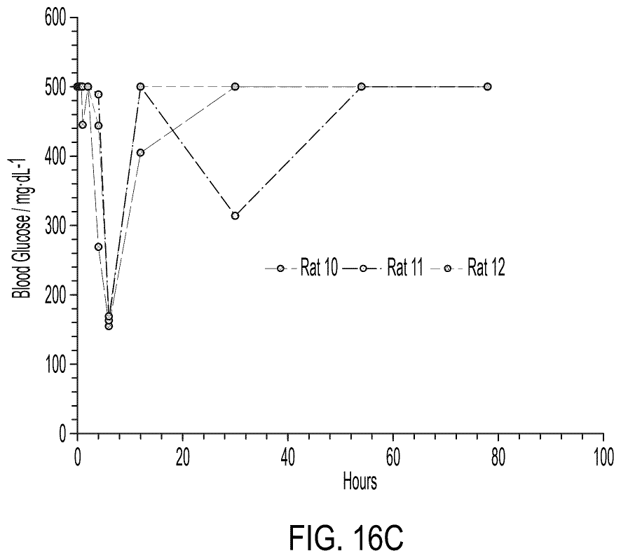

### Example 15: Insulin Release Profile for Formulation F-11 (FIG. 16A, FIG. 16B, and FIG. 16C)

The insulin release of subcutaneously injected insulin using formulation F-11 (Table 3) on diabetic rats was also studied. It was observed that subcutaneously injected 150 mg ISFGSs containing 42 IU insulin decreased the blood glucose level in diabetic rats for 30 hours. The ISFGSs were made of 1 wt % PLGA 50/50 (i.v. 0.67), 1 wt % PLA (i.v. 0.63), 4 wt % PLA (i.v. 0.47), 30 wt % meta-cresol, and 1 wt % insulin.

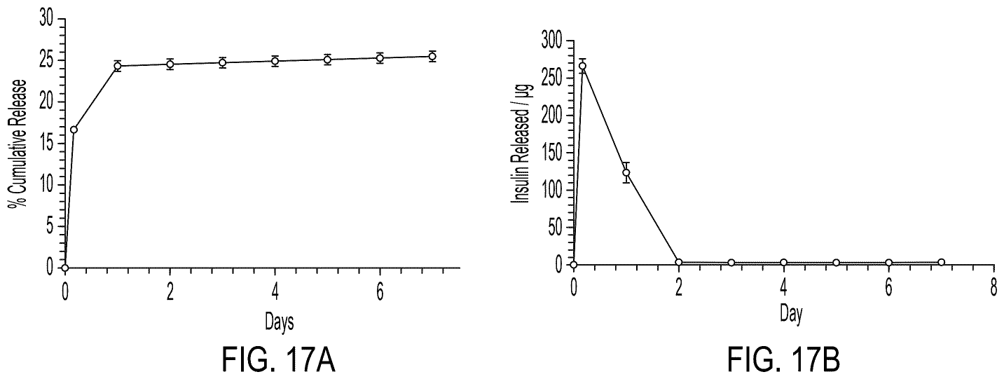

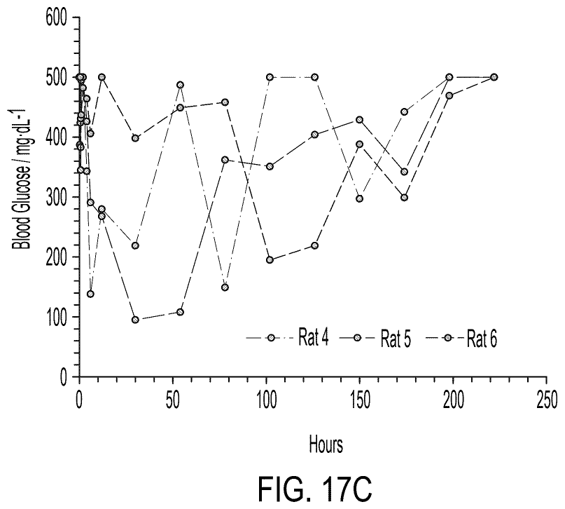

### Example 16: Insulin Release Profile for Formulation F-13 (FIG. 17A, FIG. 17B, and FIG. 17C)

The insulin release of subcutaneously injected insulin using formulation F-13 (Table 3) on diabetic rats was also studied. It was observed that subcutaneously injected 150 mg ISFGSs containing 42 IU insulin decreased the blood glucose level in diabetic rats for 7.25 days. The ISFGSs were made of 12 wt % PLGA 50/50 (i.v. 0.67), 30 wt % meta-cresol, and 1 wt % insulin.

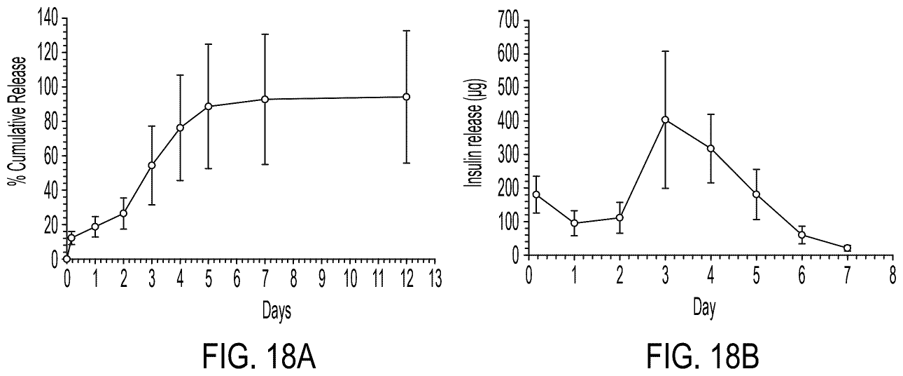

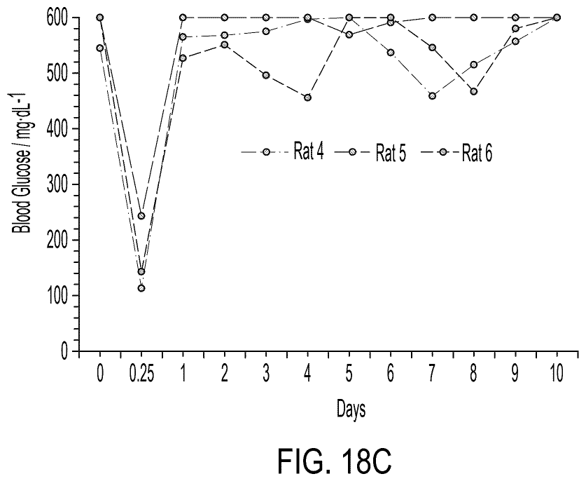

### Example 17: Insulin Release Profile for Formulation F-20 (FIG. 18A, FIG. 18B, and FIG. 18C)

The insulin release of subcutaneously injected insulin using formulation F-20 (Table 3) on diabetic rats was also studied. It was observed that subcutaneously injected 150 mg ISFGSs containing 42 IU insulin decreased the blood glucose level in diabetic rats for 9 days with fluctuation. The ISFGSs were made of 12 wt % PLGA 50/50 (i.v. 0.09), 30 wt % meta-cresol, and 1 wt % insulin.

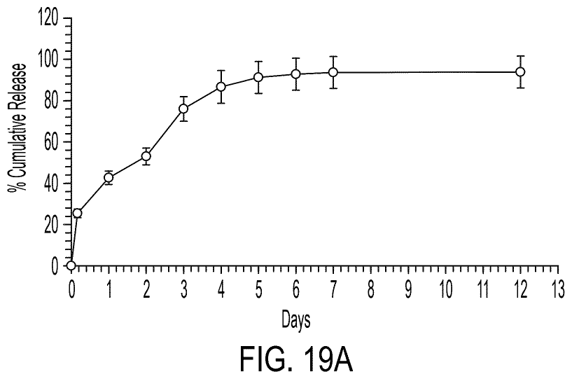

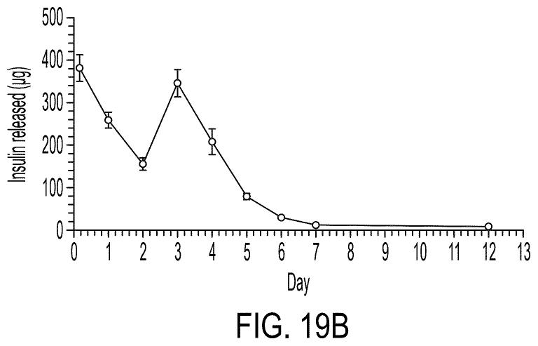

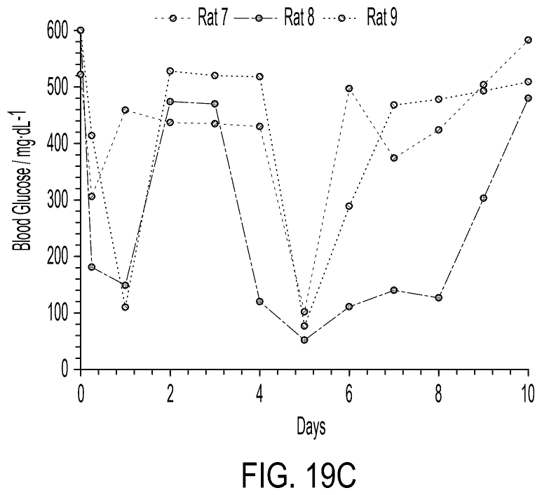

### Example 18: Insulin Release Profile for Formulation F-21 (FIG. 19A, FIG. 19B, and FIG. 19C)

The insulin release of subcutaneously injected insulin using formulation F-21 (Table 3) on diabetic rats was also studied. It was observed that subcutaneously injected 150 mg ISFGSs containing 42 IU insulin decreased the blood glucose level in diabetic rats for 9 days with fluctuation. The ISFGSs were made of 12 wt % PLGA 50/50 (i.v. 0.09), 30 wt % meta-cresol, 1 wt % insulin, and zinc at 1:28 w:w.

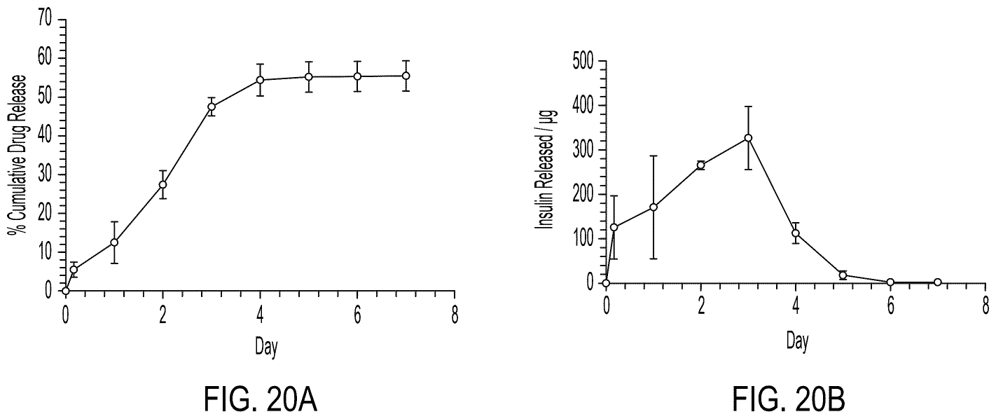

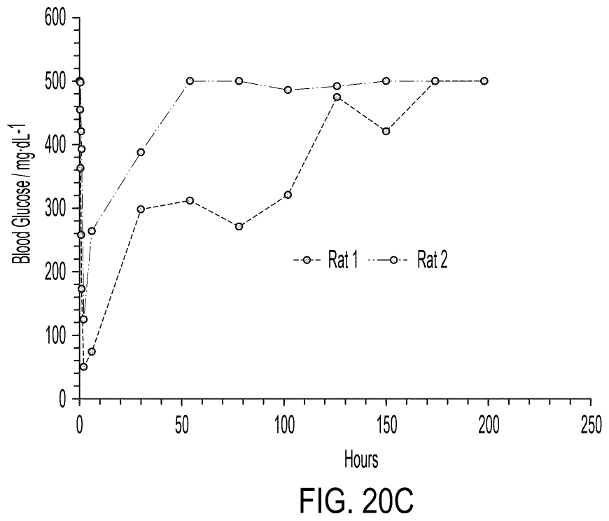

### Example 19: Insulin Release Profile for Formulation F-9 (FIG. 20A, FIG. 20B, and FIG. 20C)

The insulin release of subcutaneously injected insulin using formulation F-9 (Table 3) on diabetic rats was also studied. It was observed that subcutaneously injected 150 mg ISFGSs containing 42 IU insulin decreased the blood glucose level in diabetic rats for 6.25 days with fluctuation. The ISFGSs were made of 4 wt % PLGA 85/15 (i.v. 1.53), 4 wt % PLA (i.v. 0.63), 16 wt % PLA (i.v. 0.47), 30 wt % meta-cresol, and 1 wt % insulin.

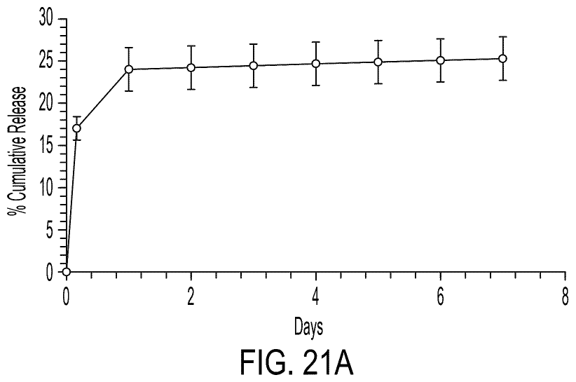

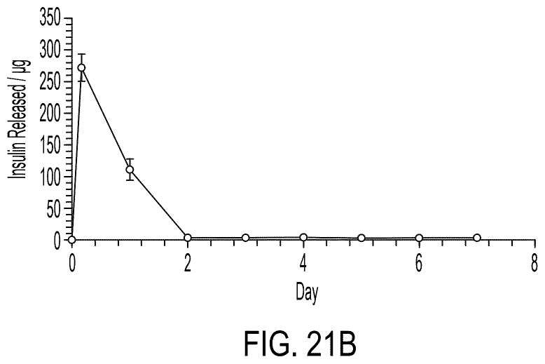

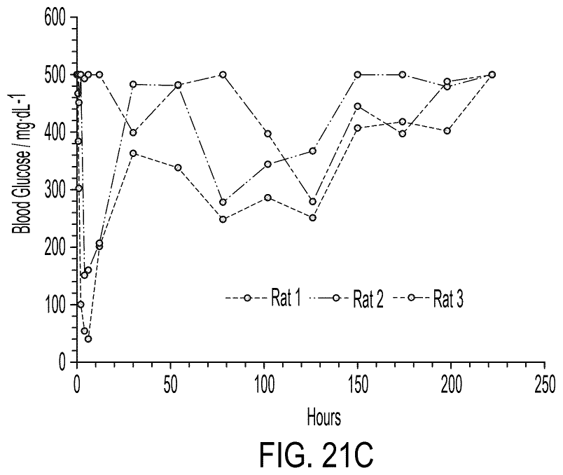

### Example 20: Insulin Release Profile for Formulation F-12 (FIG. 21A, FIG. 21B, and FIG. 21C)

The insulin release of subcutaneously injected insulin using formulation F-12 (Table 3) on diabetic rats was also studied. It was observed that subcutaneously injected 150 mg ISFGSs containing 42 IU insulin decreased the blood glucose level in diabetic rats for 8.25 days with fluctuation. The ISFGSs were made of 2 wt % PLGA 85/15 (i.v. 1.53), 2 wt % PLA (i.v. 0.63), 8 wt % PLA (i.v. 0.47), 30 wt % meta-cresol, and 1 wt % insulin.

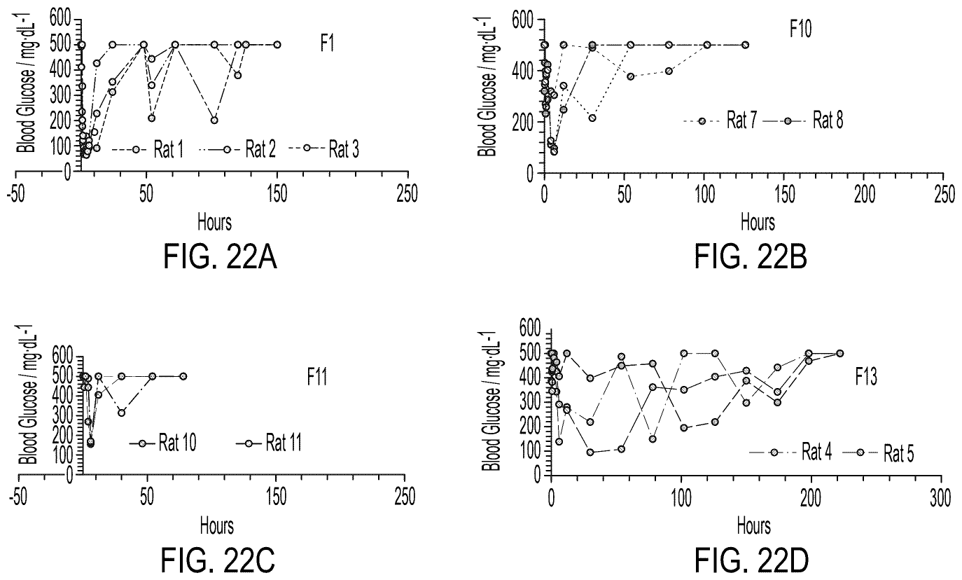

### Example 21: Effect of Injection Amount and Polymer Content on Blood Glucose Levels (FIG. 22A, FIG. 22B, FIG. 22C, and FIG. 22D)

The effects of injection amount and polymer content on the blood glucose level lowering after subcutaneously injection of ISFGSs was also studied for the following formulations from Table 3:
F1: F1: 1 wt % insulin, 2 wt % PLGA 50/50 (i.v. 0.67), 2 wt % PLA (i.v. 0.63), 8 wt % PLA (i.v. 0.47)
F10: 2 wt % insulin, 2 wt % PLGA 50/50 (i.v. 0.67), 2 wt % PLA (i.v. 0.63), 8 wt % PLA (i.v. 0.47)
F11: 1 wt % insulin, 1 wt % PLGA 50/50 (i.v. 0.67), 1 wt % PLA (i.v. 0.63), 4 wt % PLA (i.v. 0.47)
F13: 1 wt % insulin, 12 wt % PLGA 50/50 (i.v. 0.67).

The ISFGSs were made of F1 at 150 mg: 1 wt % PLGA 50/50 (i.v. 0.67), 1 wt % PLA (i.v. 0.63), 4 wt % PLA (i.v. 0.47), 30 wt % meta-cresol, and 1 wt % insulin; double amount (F10 at 300 mg); half of polymer amount (F11), and without PLA (F13). The formulations had the best blood glucose level lowering effect without PLA and with a total amount of 150 mg (F13).

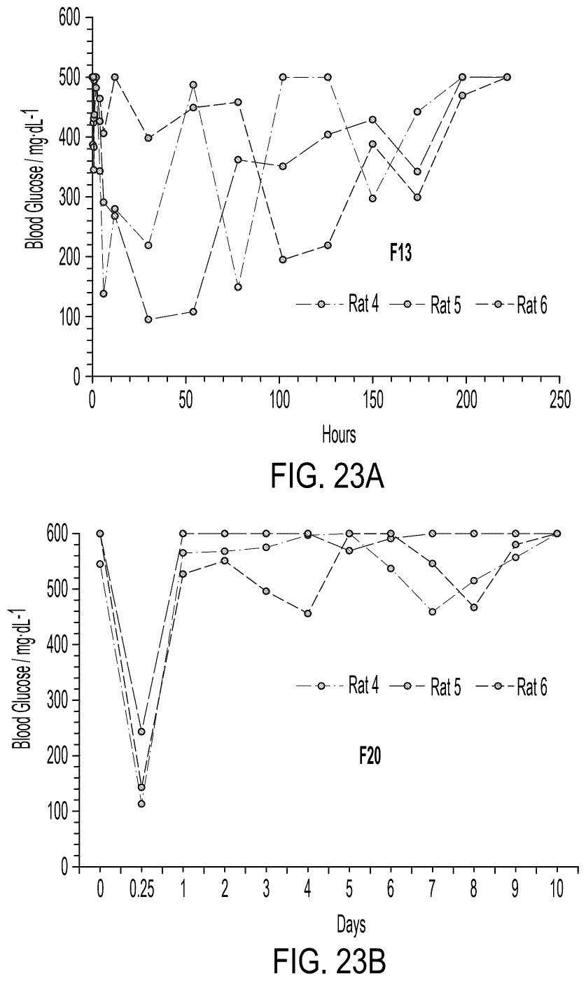

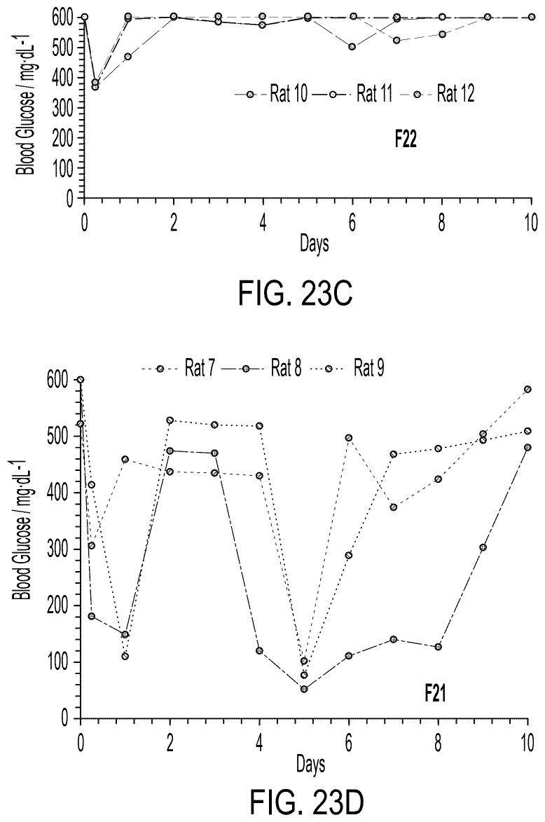

### Example 22: Effect of Injection PLGA Intrinsic Viscosity and Addition of Chitosan or Zinc on Blood Glucose Levels (FIG. 23A, FIG. 23B, FIG. 23C, and FIG. 23D)

The effects of PLGA intrinsic viscosity and addition of chitosan or zinc on blood glucose level lowering after subcutaneously injection of ISFGSs was also studied for the following formulations from Table 3:
F13: 1 wt % insulin, 12 wt % PLGA 50/50 (i.v. 0.67)
F20: 1 wt % insulin, 12 wt % PLGA 50/50 (i.v. 0.09)
F22: 1 wt % insulin, 12 wt % PLGA 50/50 (i.v. 0.09), 0.4 wt % chitosan
F21: 1 wt % insulin, 12 wt % PLGA 50/50 (i.v. 0.09), insulin:zinc=1:28 w:w.

The ISFGSs were made of 12 wt % PLGA 50/50 with i.v. 0.67 (F13) or 0.09 (F20), and with and without 0.4 wt % chitosan (F22) or zinc at insulin:zinc=1:28 w:w (F21), 30 wt % meta-cresol, and 1 wt % insulin; double amount (F10 at 300 mg); half of polymer amount (F11), and without PLA (F13) at a total weight of 150 mg. The formulations had the best blood glucose level lowering effect with PLGA i.v. 0.67 (F20 vs. F20) and zinc (F20 vs. F21 and F22).

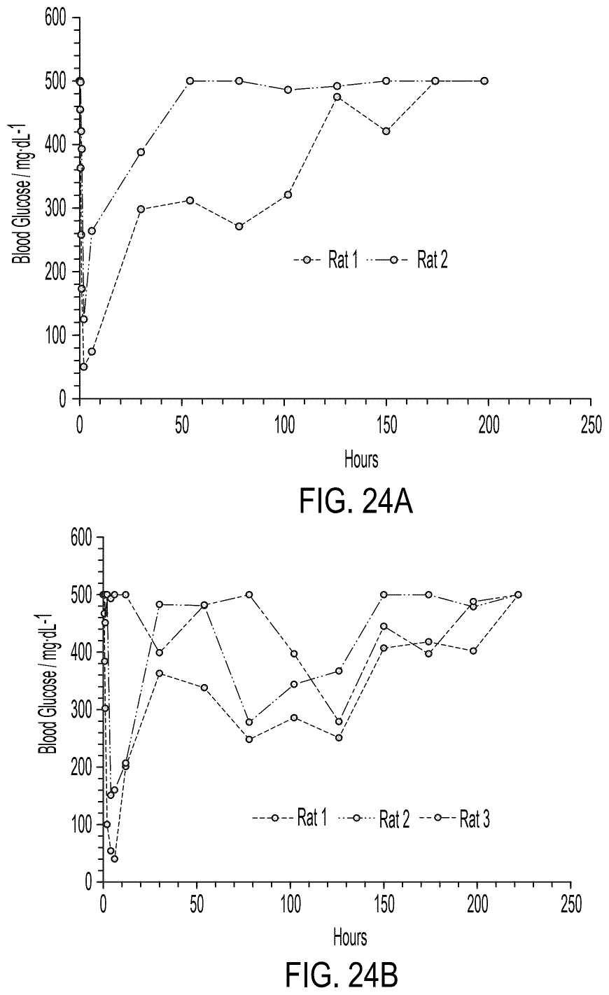

### Example 23: Effect of Polymer Content on Blood Glucose Levels (FIG. 24A and FIG. 24B)

The effects of polymer content on blood glucose level lowering after subcutaneously injection of ISFGSs was also studied for the following formulations from Table 3:
F9: 1 wt % insulin, 4 wt % PLGA 85/15 (i.v. 1.53), 4 wt % PLA (i.v. 0.63), 16 wt % PLA (i.v. 0.47)
F12: 1 wt % insulin, 2 wt % PLGA 85/15 (i.v. 1.53), 2 wt % PLA (i.v. 0.63), 8 wt % PLA (i.v. 0.47)

The ISFGSs were made of F9 at 150 mg: 4 wt % PLGA 85/15 (i.v. 1.53), 4 wt % PLA (i.v. 0.63), 16 wt % PLA (i.v. 0.47), 30 wt % meta-cresol, and 1 wt % insulin; and half polymer content (F12). The formulations had the best blood glucose level lowering effect for 8 days with the half polymer content (F12).

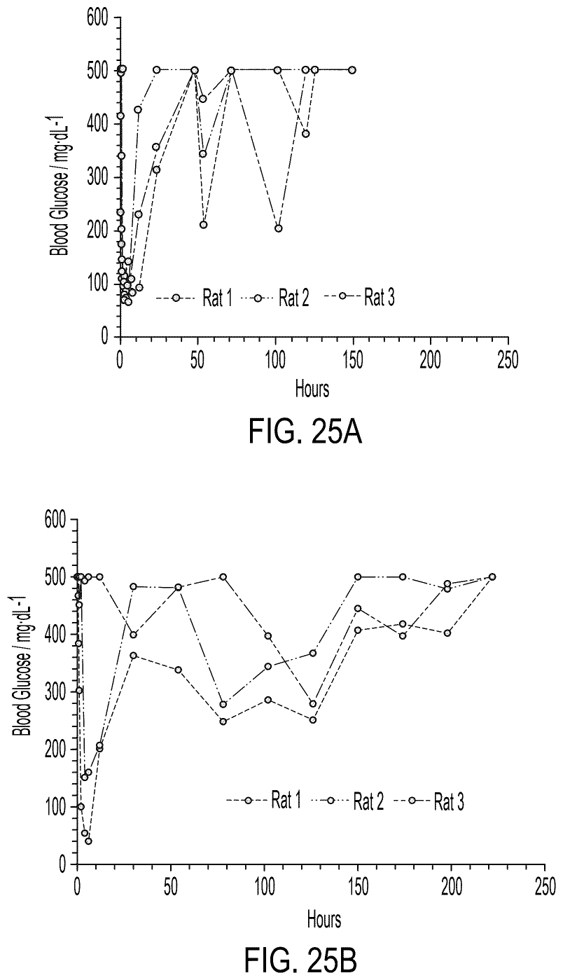

### Example 24: Effect of PLGA on Blood Glucose Levels (FIG. 25A and FIG. 25B)

The effects of PLGA on blood glucose level lowering after subcutaneously injection of ISFGSs was also studied for the following formulations from Table 3:
F1: 1 wt % insulin, 2 wt % PLGA 50/50 (i.v. 0.67), 2 wt % PLA (i.v. 0.63), 8 wt % PLA (i.v. 0.47)
F12: 1 wt % insulin, 2 wt % PLGA 85/15 (i.v. 1.53), 2 wt % PLA (i.v. 0.63), 8 wt % PLA (i.v. 0.47)

The ISFGSs were made of 2 wt % PLA (i.v. 0.63), 8 wt % PLA (i.v. 0.47), 30 wt % meta-cresol, and 1 wt % insulin with 2 wt % PLGA 50/50 (I.V. 0.67, F1) or PLGA 85/15 (i.v. 1.53, F12). The formulations had better blood glucose level lowering effect for 8 days with PLGA 85/15 (F12) than PLGA 50/50 (F1).

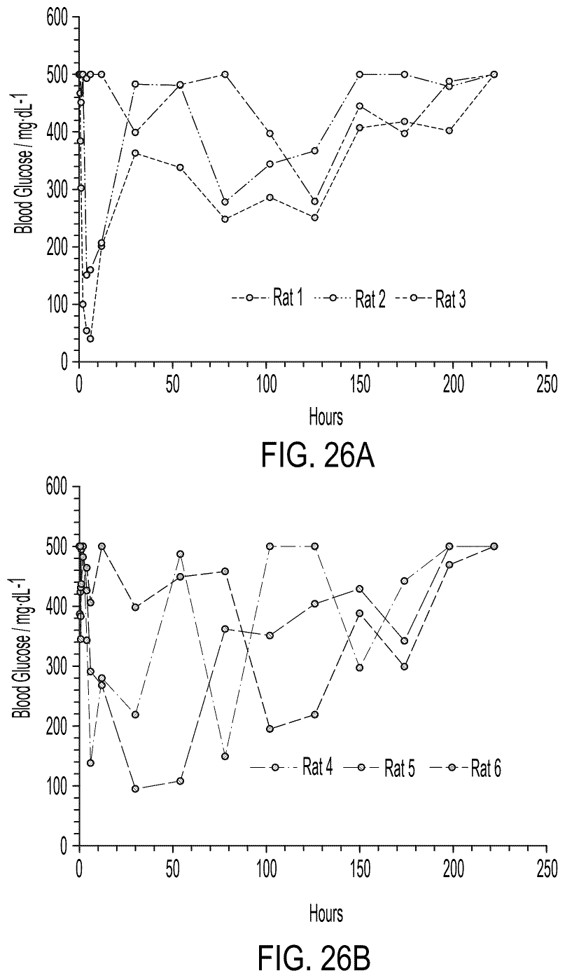

### Example 25: Effect of PLGA and PLA on Blood Glucose Levels (FIG. 26A and FIG. 26B)

The effects of PLGA and PLA on blood glucose level lowering after subcutaneously injection of ISFGSs was also studied for the following formulations from Table 3:
F12: 1 wt % insulin, 2 wt % PLGA 85/15 (i.v. 1.53), 2 wt % PLA (i.v. 0.63), 8 wt % PLA (i.v. 0.47)
F13: 1 wt % insulin, 12 wt % PLGA 50/50 (i.v. 0.67)

The ISFGSs were made of 150 mg F12 containing 2 wt % PLGA 85/15 (i.v. 1.53), 2 wt % PLA (i.v. 0.63), 8 wt % PLA (i.v. 0.47), 30 wt % meta-cresol, and 1 wt % insulin; and F13 containing 12 wt % PLA (i.v. 0.63), 30 wt % meta-cresol, and 1 wt % insulin. Both the formulations had good blood glucose level lowering effect for 8 days with F13 slightly better performance than F12.

### Summary of Results for Examples 4-25

Insulin released from some formulations (F12, F13, F21) can lower glucose level for one week in diabetic rats. F20 vs. F21: both contain 1 wt % insulin, 12 wt % PLGA 50/50 (i.v. 0.09), F21 performed better than F20 indicating zinc (insulin:zinc=1:28 w:w) is important. Formulations containing 2 wt % insulin performed better than those containing 1 wt % insulin. F1 vs. F11 and F9 vs. F12: F1 and F12 performed better than F11 and F1, respectively, indicating regarding the total polymer amount, 12 wt % was better than 6 wt % and 24 wt %. F1 vs. F12: F12 performed better than F1 possibly because PLGA 85/15 (i.v. 1.53) blended better with PLAs than PLGA 50/50 (i.v. 0.67). The in vitro insulin release and the in vivo glucose level lowering did not match probably because the in situ forming gels degraded faster in vivo than in vitro due to the presence of enzymes.

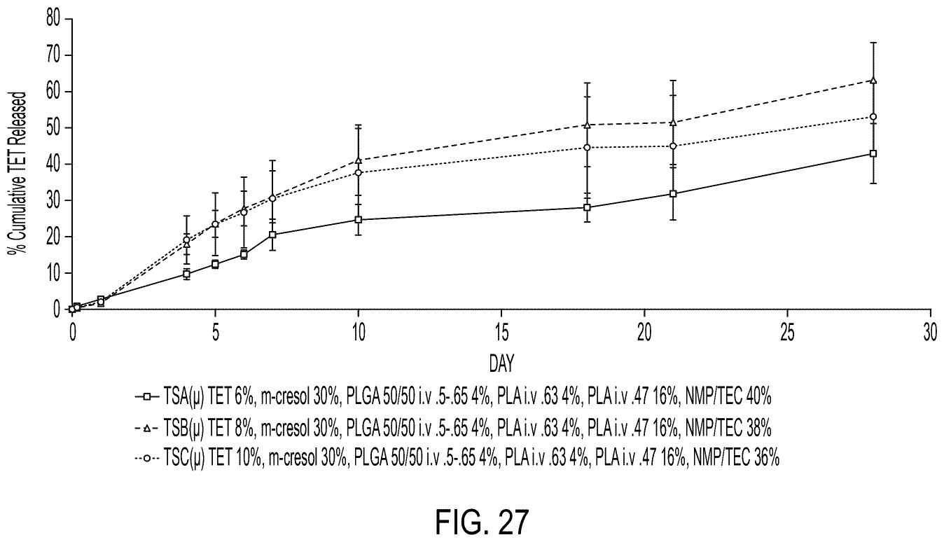

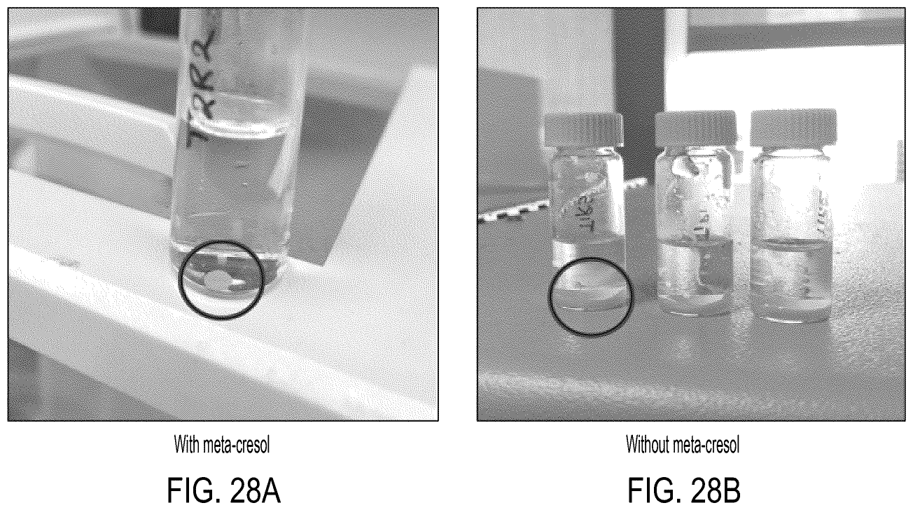

### Example 26: Tetrandrine Release Study (FIG. 27 and FIG. 28)

The effects of drug loading on the cumulative release of tetrandrine were also studied for the following three formulations:
TSA (μ) TET 6%, m-cresol 30%, PLGA 50/50 i.v 0.5-0.65 4%, PLA i.v 0.63 4%, PLA i.v 0.47 16%, NMP/TEC 40%
TSA (μ) TET 8%, m-cresol 30%, PLGA 50/50 i.v 0.5-0.65 4%, PLA i.v 0.63 4%, PLA i.v 0.47 16%, NMP/TEC 38%
TSA (μ) TET 10%, m-cresol 30%, PLGA 50/50 i.v 0.5-0.65 4%, PLA i.v 0.63 4%, PLA i.v 0.47 16%, NMP/TEC 36%

The ISFGSs were made of 150 mg: 4 wt % PLGA 50/50 (i.v. 0.5-0.65), 4 wt % PLA (i.v. 0.63), 16 wt % PLA (i.v. 0.47), 30 wt % meta-cresol, and 6, 8 or 10 wt % tetrandrine. All the three formulations could sustain the release of tetrandrine for at least 28 days, and the formulation containing 6 wt % tetrandrine had the slowest drug release kinetics. Meta-cresol in the formulations made the depot have later release profiles.

As may be observed upon reference to FIGS. 28A and 28B, meta-cresol in the formulations made the depot to have better controlled shape.

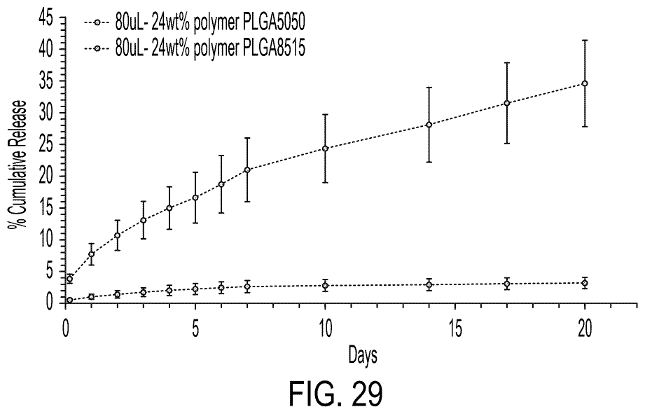

### Example 27: Dexamethasone Release Study (FIG. 29)

Example 26 demonstrates a sustained release of dexamethasone from the in situ gelling system. Thus, depot formulations can be evaluated in vivo for determining biotherapeutic effect against inflammation. Successful development of in situ depots with dexamethasone will provide progress towards an accessible treatment option for infected patients and an understanding of therapeutic efficacy of anti-inflammation therapy in a sustained release dosage form for arthritis, asthma, skin diseases, eye problems, breathing problems, bone marrow problems, kidney problems, cancers, hearing loss, immune system disorders, blood/hormone disorders, bowel disorders, adrenal gland disorders, Cushing's syndrome, flare-ups of multiple sclerosis, and inflammation response and mortality associated with COVID-19 cytokine storm.

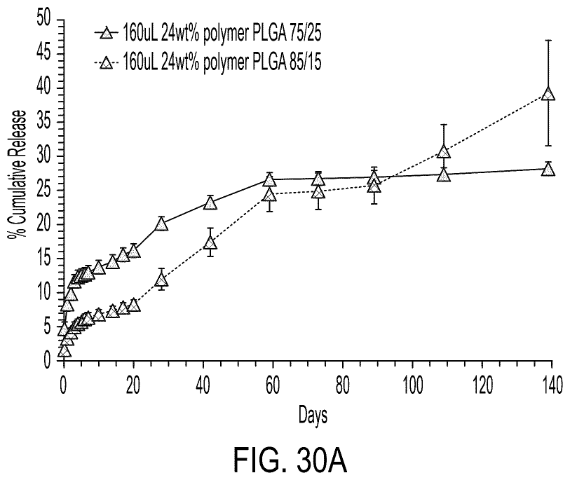

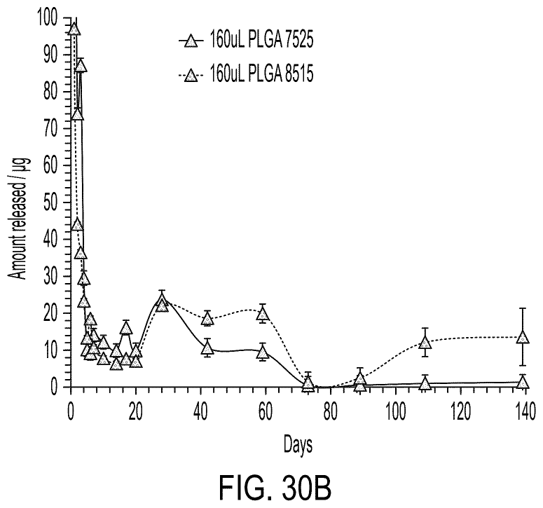

### Example 28: Remdesivir Release Study (FIG. 30)

Example 27 demonstrates a sustained release of antiviral agent remdesivir from the in situ gelling system. Thus, depot formulations can be evaluated in vivo for determining biotherapeutic effect against COVID-19. Successful development of in situ depots with remdesivir will provide progress towards an accessible treatment option for infected patients and an understanding of therapeutic efficacy of anti-viral therapy in a sustained release dosage form for COVID-19.

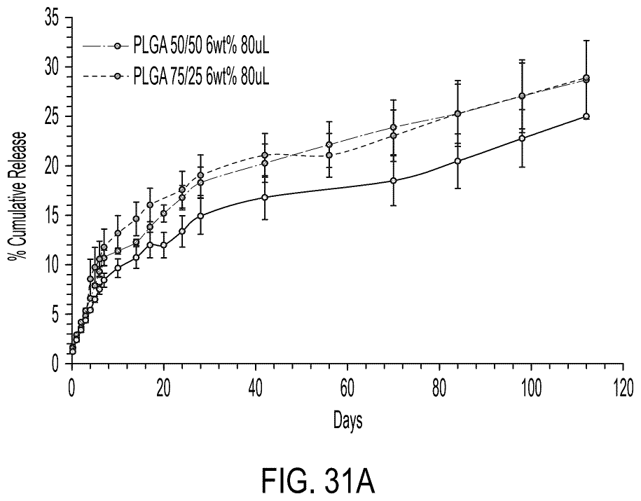

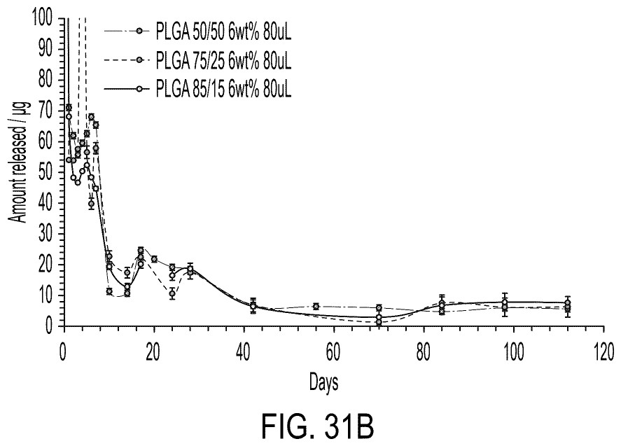

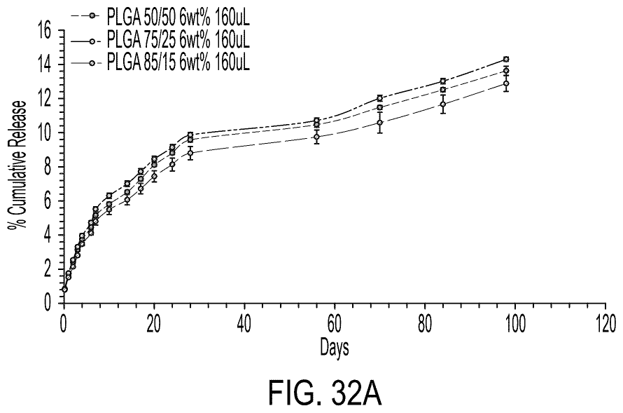

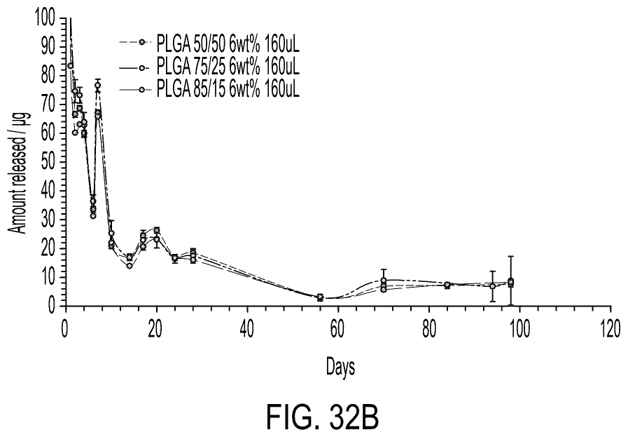

### Example 29: GS-441524 Release Study (FIG. 31 and FIG. 32)

Example 28 demonstrates a sustained release of antiviral agent GS-441524 from the in situ gelling system. Thus, depot formulations can be evaluated in vivo for determining biotherapeutic effect against COVID-19. Successful development of in situ depots with GS-441524 will provide progress towards an accessible treatment option for infected patients and an understanding of therapeutic efficacy of anti-viral therapy in a sustained release dosage form for COVID-19.

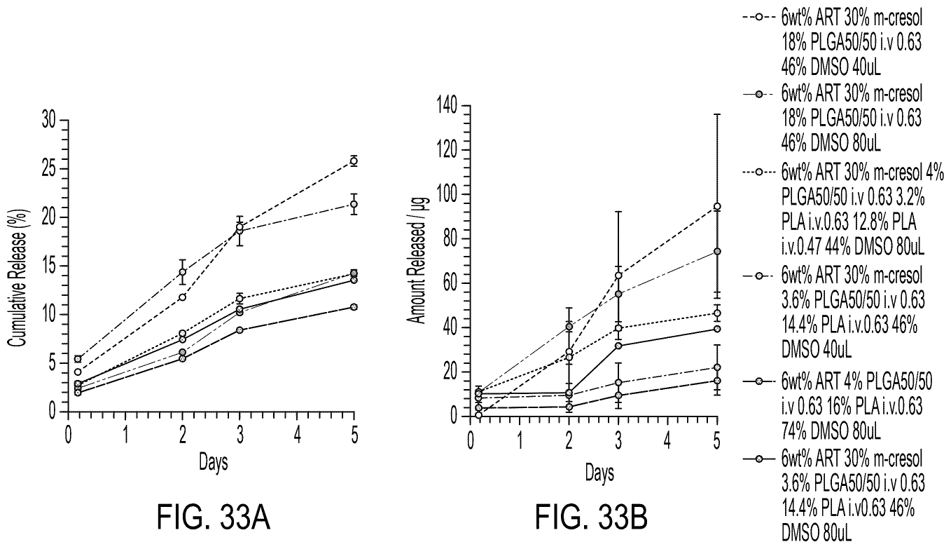

### Example 30: Artemisinin Release Study (FIG. 33)

Example 29 demonstrates a sustained release of artemisinin from the in situ gelling system. Thus, depot formulations can be evaluated in vivo for determining biotherapeutic effect against malaria and leukemias. Successful development of in situ depots with artemisinin will provide progress towards an accessible treatment option for malarial and cancer patients and an understanding of therapeutic efficacy of anti-malarial and anti-cancer therapies in a sustained release dosage form for malaria and leukemias.

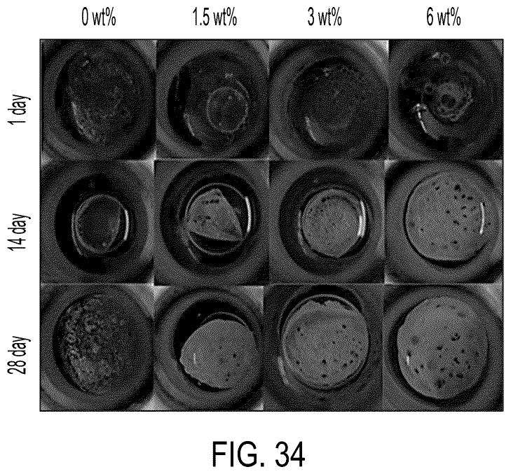

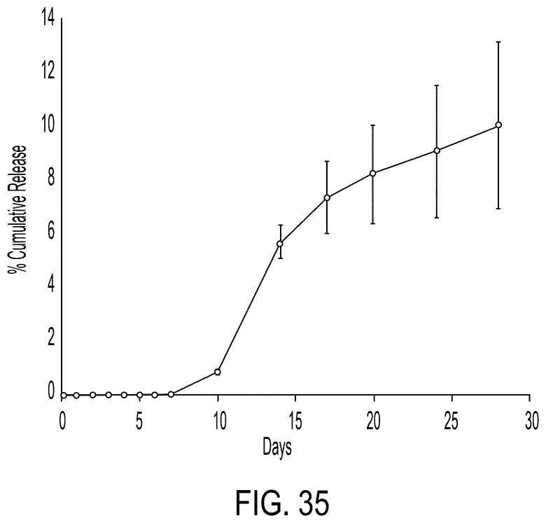

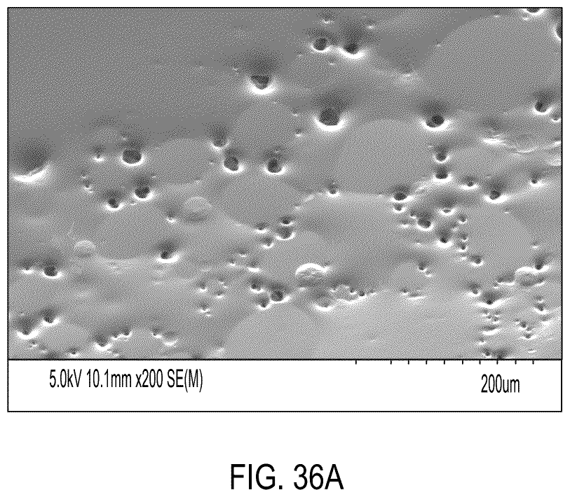

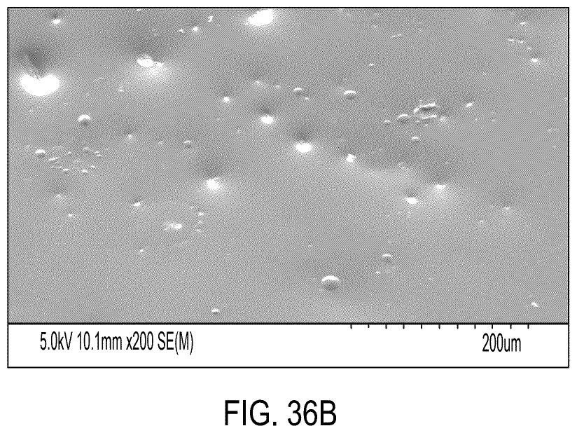

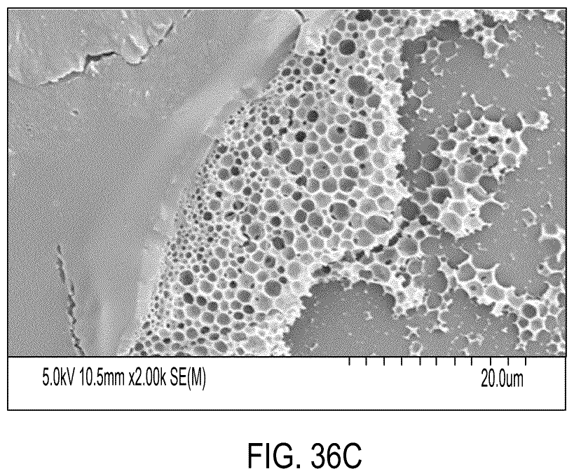

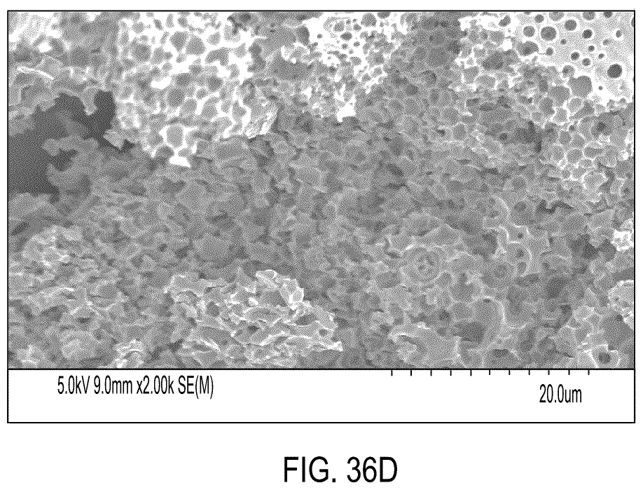

### Example 31: ART838 Release Study (FIG. 34, FIG. 35, and FIG. 36)

The appearance of the depot is shown in FIG. 34. ISFGS turbidity increased with time and with ART838 loading content, and all loaded ISDs became by day 28. See FIG. 35. The diffusion of the solvents from the depot in the first week was not sufficient for ART838 to diffuse out of the ISFGS due to the hydrophobic interactions between ART838 and PLGA/PLA polymers. The ISFGS system is able to continually release ART838 for 1 month and the ISD form can be tuned to control ART838 release profiles. See FIG. 36.

The results in FIG. 34 and FIG. 35 demonstrate that ISFGSs can provide sustained release of ART838 into PBS over days-weeks. This delayed release profile might be beneficial to potentiate antitumor responses initiated by other drug agents. Therefore it is contemplated that the polymeric formulations of the ISFGS system can be altered to tune the ART838 release curve, aiming to modulate the initiation of release while maintaining the stable release observed here. In addition, the ISFGSs can be used with other dimeric ART analogs or other drug compounds, both for administration and for research purposes, for example to analyze cytotoxicity and bioeffects of the loaded ISFGSs using in vitro cell culture models.

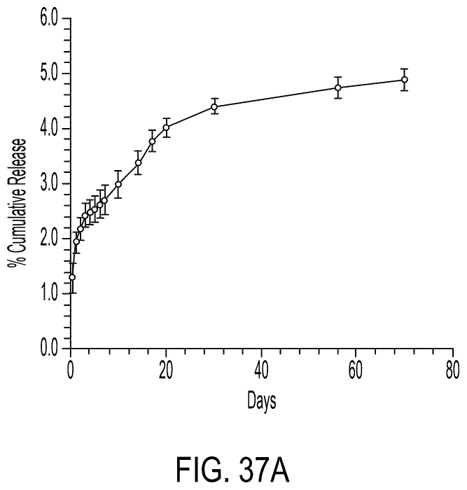

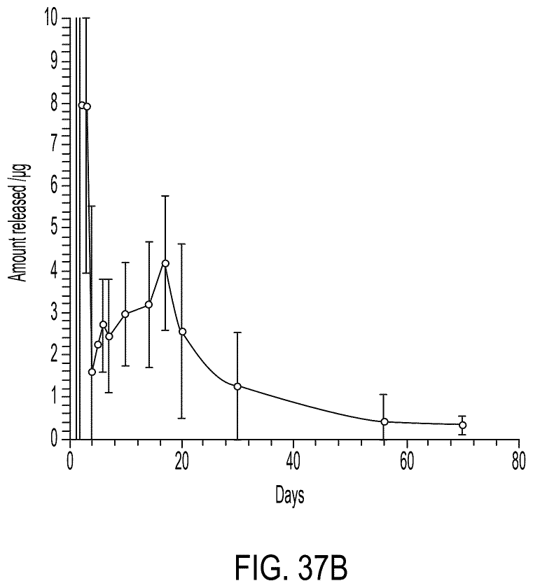

### Example 32: Gilteritinib Release Study (FIG. 37)

Example 31 demonstrates a sustained release of gilteritinib from the in situ gelling system. Thus, depot formulations can be evaluated in vivo for determining biotherapeutic effect against leukemias. Successful development of in situ depots with artemisinin will provide progress towards an accessible treatment option for cancer patients and an understanding of therapeutic efficacy of anti-cancer therapies in a sustained release dosage form for leukemias.

### REFERENCES

All references listed below and throughout the specification are hereby incorporated by reference in their entirety.
1. WHO Coronavirus Dashboard 2023; covid19.who.int/
2. Rahmah L, Abarikwu S O, Arero A G, et al. Pharmacol Rep. 2022; 74 (6): 1326-1327.
3. Pandemic “Mapping our unvaccinated word” 2023; pandem-ic.com/mapping-our-unvaccinated-world/.
4. Humeniuk, R., Mathias, A., Kirby, B. J. et al. Clinical Pharmacokinetics 2021; 60:569-583.
5. The RECOVERY Collaborative Group. N Engl J Med 2021; 384:693-704.
6. Posner, G. H. and Mott, B. T. (2014). U.S. Pat. No. 9,487,538.
7. Mott B T. Bioorg & Med Chem. 2013; 21:3702-07.
8. Janagam D R. Adv Biosys. 2017; 1:1700097.
9. Janagam, D R. Pharmaceutics. 2016; 8.
10. Zhao, M. Eur J Pharmacol. 2021; 890:173646.
11. Beigel, J. H. N Engl J Med 2020; 383:1813-1826.
12. Yan, V. C. ACS Med. Chem. 2020; 11:1361-1366.

1. An injectable polymer matrix drug delivery system comprising:
a) a biodegradable polymer selected from the group consisting of polyester, poly(lactic-co-glycolic acid), poly(lactic acid), poly(ε-caprolactone), poly(ethylene glycol-block-lactic acid), poly(alkylcyanoacrylate), polyanhydride, poly(bis(pcarboxyphenoxy) propane-sebacic acid), polyorthoester, polyphosphoester, polyphosphazene, polyurethane, and poly(amino acid), or combinations thereof;
b) a solvent or a combination of solvents;
c) an alkylphenol; and
d) a drug.

2. The drug delivery system of claim 1, wherein the biodegradable polymer is selected from poly(lactic-co-glycolic acid), poly(lactic acid), and poly(ε-caprolactone), or combinations thereof.

3. The drug delivery system of claim 1, wherein the solvent is selected from N-methyl-2-pyrrolidone (NMP), benzyl benzoate (BB), benzyl alcohol (BA), triethyl citrate (TEC), acetyl triethyl citrate (ATEC), ethyl acetate (EA), acetyl tributyl citrate (ATBC), dimethyl sulfoxide (DMSO), and any combination thereof.

4. The drug delivery system of claim 1, wherein the biodegradable polymer is selected from poly(L-lactic acid) and poly(D,L-lactic acid), or combinations thereof.

5. The drug delivery system of claim 1, wherein the alkylphenol is a cresol.

6. The drug delivery system of claim 5, wherein the cresol is m-cresol.

7. The drug delivery system of claim 1, wherein the drug is selected from the group consisting of insulin, an insulin analog, tetrandrine, dexamethasone, remdesivir, GS-441524, artemisinin, ART838, an artemisinin derivative, gilteritinib, and any combination thereof.

8. The drug delivery system of claim 1, wherein the drug is insulin.

9. The drug delivery system of claim 1, wherein the drug is an insulin analog.

10. The drug delivery system of claim 1, wherein the drug is a combination of insulin and gilteritinib.

11. The drug delivery system of claim 1, wherein the system further comprises zinc ions.

12. The drug delivery system of claim 1, wherein the solvent is selected from the group consisting of N-methyl-2-pyrrolidone (NMP), benzyl benzoate (BB), benzyl alcohol (BA), triethyl citrate (TEC), acetyl triethyl citrate (ATEC), ethyl acetate (EA) dimethyl sulfoxide (DMSO), and a combination thereof; the biodegradable polymer is selected from the group consisting of poly(L-lactic acid), poly(D,Llactic acid), and a combination thereof, and the active pharmaceutical ingredient is selected from the group consisting of insulin, an insulin analog, tetrandrine, dexamethasone, remdesivir, GS-441524, artemisinin, ART838, an artemisinin derivative, gilteritinib, and any combination thereof.

13. The drug delivery system of claim 1, wherein the solvent combination is NMP and TEC; NMP and ATEC; NMP and ATBC; NMP and BB; NMP and BA; NMP and EA; TEC and BB; ATEC and BB; ATBC and BB; TEC and BA; ATEC and BA; ATBC and BA; TEC and EA; ATEC and EA; ATBC and EA; NMP, TEC and BB; NMP, ATEC and BB; NMP, ATBC and BB; NMP, TEC and BA; NMP, ATEC and BA; NMP, ATBC and BA; NMP, TEC and EA; NMP, ATEC and EA; NMP, ATBC and EA; TEC, BB and EA; ATEC, BB and EA; ATBC, BB and EA; TEC, BA and EA; ATEC, BA and EA; ATBC, BA and EA; NMP and DMSO; BB and DMSO; BA and DMSO; TEC and DMSO; ATEC and DMSO; ATBC and DMSO; EA and DMSO; NMP, TEC and DMSO; NMP, ATEC and DMSO; NMP, ATBC and DMSO; NMP, BB and DMSO; NMP, BA and DMSO; NMP, EA and DMSO; TEC, ATEC and DMSO; TEC, BB and DMSO; TEC, BA and DMSO; TEC, EA and DMSO; ATEC, BB and DMSO; ATEC, BA and DMSO; ATEC, EA and DMSO; BB, BA and DMSO; BB, EA and DMSO; or BA, EA and DMSO.

14. The drug delivery system of claim 1, wherein the solvent combination is NMP and TEC, NMP and ATEC, NMP and BB, NMP and BA, or DMSO and TEC.

15. The drug delivery system of claim 1, wherein the system comprises the polymer in about 0-50% by weight, the solvent in about 50-95% by weight, the alkylphenol in about 0.1-50% by weight, and insulin or an insulin analog in about 0.130% by weight.

16. The drug delivery system of claim 1, wherein the system is packaged in two syringes comprising:
a) one syringe containing the polymer solution,
b) one syringe containing a solvent or a combination of solvents, the alkylphenol, and the drug.

17. The drug delivery system of claim 16, wherein the system is formulated for subcutaneous injection or intramuscular injection.

18. A method of treating diabetes mellitus in a subject in need thereof, the method comprising administering to a subject in need thereof the injectable polymer matrix drug delivery system of claim 1.

19. The method of claim 18, wherein the diabetes mellitus is type 1 diabetes or type 2 diabetes.

20. A method of reducing blood glucose levels in a subject in need thereof, the method comprising administering to a subject in need thereof the injectable polymer matrix drug delivery system of claim 1.

21. A method of treating a condition selected from the group consisting of inflammation, infection, cancer, hearing loss, COVID19, and HIV in a subject in need thereof, the method comprising administering to a subject in need thereof the injectable polymer matrix drug delivery system of claim 1.

22-27. (canceled)
